SEQUENCE LISTING (31 sequences)

SEQ ID NO: 1 - protein, 5 aa, Human
  SITE (1)...(5): cyclo Glu1-Lys5
EWLRK

SEQ ID NO: 2 - protein, 7 aa, Human
  SITE (1)...(5): cyclo Glu1-Lys5
EWLRKLL

SEQ ID NO: 3 - protein, 14 aa, Human
  SITE (5)...(9): cyclo Glu5-Lys9
MERVEWLRKLLQDV

SEQ ID NO: 4 - protein, 31 aa, Human
  SITE (22)...(26): cyclo Glu22-Lys26
XVSEIQLXHNLGXXXXXMERVEWLRKLLQDV

SEQ ID NO: 5 - protein, 31 aa, Human
  SITE (22)...(26): cyclo Glu22-Lys26
SVSEIQLMHNLGKXXXXMERVEWLRKLLQDV

SEQ ID NO: 6 - protein, 31 aa, Human
  SITE (22)...(26): cyclo Glu22-Lys26
SVSEIQLMHNLGKHKKKMERVEWLRKLLQDV

SEQ ID NO: 7 - protein, 31 aa, Human
  SITE (22)...(26): cyclo Glu22-Lys26
SVSEIQLMHNLGKHLKKMERVEWLRKLLQDV

SEQ ID NO: 8 - protein, 31 aa, Human
  SITE (22)...(26): cyclo Glu22-Lys26
SVSEIQLMHNLGKKKKKMERVEWLRKLLQDV

SEQ ID NO: 9 - protein, 31 aa, Human
  SITE (22)...(26): cyclo Glu22-Lys26
SVSEIQLMHNLGKHLKSMERVEWLRKLLQDV

SEQ ID NO: 10 - protein, 31 aa, Human
  SITE (22)...(26): cyclo Glu22-Lys26
SVSEIQLMHNLGKHLNSMERVEWLRKLLQDV

SEQ ID NO: 11 - protein, 84 aa, Human
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 12 - protein, 15 aa, Human
  SITE (6)...(10): cyclo Glu6-Lys10
CMERVEWLRKLLQDV

SEQ ID NO: 13 - protein, 16 aa, Human
  SITE (6)...(10): cyclo Glu6-Lys10
SMERVEWLRKLLQDVC

SEQ ID NO: 14 - protein, 32 aa, human
  SITE (22)...(26): cyclo Glu22-Lys26
XVSEIQLXHNLGXXXXXMERVEWLRKLLQDVH

SEQ ID NO: 15 - protein, 33 aa, human
  SITE (22)...(26): cyclo Glu22-Lys26
XVSEIQLXHNLGXXXXXMERVEWLRKLLQDVHN

SEQ ID NO: 16 - protein, 34 aa, human
  SITE (22)...(26): cyclo Glu22-Lys26
XVSEIQLXHNLGXXXXXMERVEWLRKLLQDVHNF

SEQ ID NO: 17 - protein, 32 aa, human
  SITE (22)...(26): cyclo Glu22-Lys26
SVSEIQLMHNLGKXXXXMERVEWLRKLLQDVH

SEQ ID NO: 18 - protein, 33 aa, human
  SITE (22)...(26): cyclo Glu22-Lys26
SVSEIQLMHNLGKXXXXMERVEWLRKLLQDVHN

SEQ ID NO: 19 - protein, 34 aa, human
  SITE (22)...(26): cyclo Glu22-Lys26
SVSEIQLMHNLGKXXXXMERVEWLRKLLQDVHNF

SEQ ID NO: 20 - protein, 32 aa, human
  SITE (22)...(26): cyclo Glu22-Lys26
SVSEIQLMHNLGKHKKKMERVEWLRKLLQDVH

SEQ ID NO: 21 - protein, 33 aa, human
  SITE (22)...(26): cyclo Glu22-Lys26
SVSEIQLMHNLGKHKKKMERVEWLRKLLQDVHN

SEQ ID NO: 22 - protein, 34 aa, human
  SITE (22)...(26): cyclo Glu22-Lys26
SVSEIQLMHNLGKHKKKMERVEWLRKLLQDVHNF

SEQ ID NO: 23 - protein, 32 aa, human
  SITE (22)...(26): cyclo Glu22-Lys26
SVSEIQLMHNLGKHLKKMERVEWLRKLLQDVH

SEQ ID NO: 24 - protein, 33 aa, human
  SITE (22)...(26): cyclo Glu22-Lys26
SVSEIQLMHNLGKHLKKMERVEWLRKLLQDVHN

SEQ ID NO: 25 - protein, 34 aa, human
  SITE (22)...(26): cyclo Glu22-Lys26
SVSEIQLMHNLGKHLKKMERVEWLRKLLQDVHNF

SEQ ID NO: 26 - protein, 32 aa, human
  SITE (22)...(26): cyclo Glu22-Lys26
SVSEIQLMHNLGKKKKKMERVEWLRKLLQDVH

SEQ ID NO: 27 - protein, 33 aa, human
  SITE (22)...(26): cyclo Glu22-Lys26
SVSEIQLMHNLGKKKKKMERVEWLRKLLQDVHN

SEQ ID NO: 28 - protein, 34 aa, human
  SITE (22)...(26): cyclo Glu22-Lys26
SVSEIQLMHNLGKKKKKMERVEWLRKLLQDVHNF

SEQ ID NO: 29 - protein, 32 aa, human
  SITE (22)...(26): cyclo Glu22-Lys26
SVSEIQLMHNLGKHLKSMERVEWLRKLLQDVH

SEQ ID NO: 30 - protein, 33 aa, human
  SITE (22)...(26): cyclo Glu22-Lys26
SVSEIQLMHNLGKHLKSMERVEWLRKLLQDVHN

SEQ ID NO: 31 - protein, 34 aa, human
  SITE (22)...(26): cyclo Glu22-Lys26
SVSEIQLMHNLGKHLKSMERVEWLRKLLQDVHNF

(19) United States
(12) Patent Application Publication
(10) Pub. No.: US 2006/0068444 A1
(43) Pub. Date: Mar. 30, 2006

(54) Compositions and methods for detecting cyclic analogs of hPTH

(75) Inventors: Paul Morley, Ottawa (CA); Ping Gao, San Diego, CA (US); Gordon Edward Willick, Orleans (CA)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: Zelos Therapeutics, Inc, Ottawa (CA); National Research Council of Canada, Ottawa (CA)

(21) Appl. No.: 11/083,063

(22) Filed: Mar. 17, 2005

Related U.S. Application Data
(60) Provisional application No. 60/554,777, filed on Mar. 19, 2004.

(51) Int. Cl. C07K 16/26; G01N 33/53

(52) U.S. Cl. 435/7.1; 530/388.24

(57) ABSTRACT

The present invention is directed to novel antigens, antibodies and antigen binding fragments thereof, and to immunoassays and kits using these antigens and antibodies. The compositions and methods are useful in the determination of levels of cyclic analogs of hPTH in sample fluids, such as serum or plasma. The antibodies and methods of the invention have the particular advantage of possessing binding specificity for cyclic analogs of hPTH.

### RELATED APPLICATION(S)

This application claims the benefit of U.S. Provisional Application No. 60/554,777, filed on Mar. 19, 2004. The entire teachings of the above application(s) are incorporated herein by reference.

### BACKGROUND OF THE INVENTION

Osteoporosis is a leading cause of disability in the elderly, particularly elderly women. It is a progressive disease which results in the reduction of total bone mass and increased bone fragility. This often results in spontaneous fractures of load-bearing bones, and the physical and mental deterioration characteristic of immobilizing injuries. Post-menopausal osteoporosis is caused by the disappearance of estrogens, which triggers a decade-long acceleration of bone turnover with an increased imbalance between resorption of old bone and formation of new bone. This results in thinning, increased porosity, and trabecular depletion of load-bearing bones. Osteoporosis is also associated with hyperthyroidism, hyperparathyroidism, Cushing's syndrome, and the use of certain steroidal drugs. Remedies historically have involved increased dietary calcium, estrogen therapy, vitamin D, and most commonly, treatment with agents such as antiresorptives that inhibit bone resorption by osteoclasts.

It has recently been realized that human parathyroid hormone (hPTH) and certain analogs of hPTH are stimulators of bone growth, and are thus useful in the treatment of osteoporosis. hPTH is a hypercalcemic hormone, produced by the parathyroid gland which functions to elevate blood calcium levels. When serum calcium is reduced to below a normal level, the parathyroid gland releases hPTH and the calcium level is increased. The mechanisms by which the calcium levels are increased include: resorption of bone calcium, increased absorption of calcium from the intestine, and increased renal absorption of calcium from nascent urine in the kidney tubules. Although continuously infused low levels of hPTH can remove calcium from bone, the same low doses when intermittently injected can actually promote bone growth.

hPTH operates through activation of two secondary messenger systems, G_{S}-protein activated adenylyl cyclase (AC) and G_{q}-protein activated phospholipase C_{β}. The native HPTH sequence has been shown to have all of these activities. The G_{q}-protein activated phospholipase C_{β} which results in a stimulation of membrane-bound protein kinase Cs (PKC) activity, has been shown to require hPTH residues 29 to 32 (Jouishomme et al (1992) Endocrinology 130(1): 53-60). It has been established that the increase in bone growth, i.e., that effect which is useful in the treatment of osteoporosis, is coupled to the ability of the peptide sequence to increase AC activity.

The linear analogue, hPTH-(1-31)-NH₂, has only AC stimulating activity and has been shown to be fully active in the restoration of bone loss in the ovariectomized rat model (Rixon, R. H. et al. (1994) J. Bone Miner. Res. 9:1179-1189; Whitfield et al. (1996) Calcified Tissue Int. 58: 81-87; Willick et al., U.S. Pat. No. 5,556,940). U.S. Pat. No. 5,955,425, discloses cyclic analogs of hPTH-(1-31). These analogs have lactams formed for example between either Glu²² and Lys²⁶ or Lys²⁶ and Asp³⁰. In addition, the natural Lys²⁷ is substituted by either a Leu or other hydrophobic residue, such as Ile, norleucine, Met, Val, Ala, Trp, or Phe. These analogs show enhanced activities in bone restoration and bioavailabilities with respect to the linear analogs, without producing a significant increase in the circulating calcium levels.

A need exists for methods which detect such analogs.

### SUMMARY OF THE INVENTION

The present invention is directed to novel antigens, antibodies or antigen binding fragments thereof, and to assays (e.g., immunoassays) and kits using these antigens and antibodies. These antigens, antibodies, immunoassays, and kits are useful in the determination of levels of cyclic analogs of HPTH in sample fluids, such as serum or plasma.

In one embodiment, the present invention is an antibody (one or more) or antigen binding fragment thereof which has binding specificity for a cyclic analog of human parathyroid hormone (hPTH).

In another embodiment, the present invention is an antibody (one or more) or antigen binding fragment thereof which has binding specificity for a cyclic analog of human parathyroid hormone (hPTH), wherein the cyclic analog comprises an amino acid sequence: Glu-Trp-Leu-Arg-Lys (SEQ ID NO: 1) which is cyclized between Glu¹ and Lys⁵.

In another embodiment, the present invention is an antibody (one or more) or antigen binding fragment thereof which has binding specificity for a cyclic analog of human parathyroid hormone (hPTH), wherein the cyclic analog comprises an amino acid sequence: Glu-Trp-Leu-Arg-Lys-Leu-Leu (SEQ ID NO: 2) which is cyclized between Glu¹ and Lys⁵.

In another embodiment, the present invention is an antibody (one or more) or antigen binding fragment thereof which has binding specificity for a cyclic analog of human parathyroid hormone (hPTH), wherein the cyclic analog comprises an amino acid sequence: Met-Glu-Arg-Val-Glu-Trp-Leu-Arg-Lys-Leu-Leu-Gln-Asp-Val (SEQ ID NO: 3) which is cyclized between Glu⁵ and Lys⁹.

In another embodiment, the present invention is an antibody (one or more) or antigen binding fragment thereof which has binding specificity for a cyclic analog of human parathyroid hormone (hPTH), wherein the cyclic analog comprises an amino acid sequence: R-NH-Xaa1-Val-Ser-Glu-Ile-Gln-Leu-Xaa8-His-Asn-Leu-Gly-Xaa13-Xaa 14-Xaa 115-Xaa 16-Xaa 17-Met-Glu-Arg-Val-Glu-Trp-Leu-Arg-Lys-Leu-Leu-Gln-Asp-Val-Y (SEQ ID NO: 4), wherein: the cyclic analog is cyclized between Glu²² and Lys²⁶; R is a hydrogen or any linear or branched chain alkyl, acyl or aryl group; Xaa1 is serine, alanine, norleucine, or α-aminoisobutyric acid; Xaa8 is methionine, norisoleucine, norleucine, or a hydrophobic amino acid; Xaa13 is lysine, ornithine, glutamic acid, aspartic acid, cysteine, or homocysteine; Xaa14 is histidine or a water soluble amino acid; Xaa15 is leucine or a water soluble amino acid; Xaa16 is asparagine or a water soluble amino acid; Xaa17 is serine or a water soluble amino acid; and Y is, His-X, His-Asn-X, or His-Asn-Phe-X; where X is NH₂ or OH.

In another embodiment, the present invention is an antibody (one or more) or antigen binding fragment thereof which has binding specificity for a cyclic analog of human parathyroid hormone (hPTH), wherein the cyclic analog comprises an amino acid sequence: H-NH-Ser-Val-Ser-Glu-Ile-Gln-Leu-Met-His-Asn-Leu-Gly-Lys-Xaa14-Xaa15-Xaa16-Xaa17-Met-Glu-Arg-Val-Glu-Trp-Leu-Arg-Lys-Leu-Leu-Gln-Asp-Val-Y (SEQ ID NO: 5), wherein: the cyclic analog is cyclized between Glu²² and Lys²⁶; Xaa14 is histidine or lysine; Xaa15 is leucine, lysine, or arginine; Xaa16 is asparagine, ornithine, homocitrulline, aspartic acid, arginine, lysine, d-lysine, serine, or glycine; Xaa17 is serine, glutamic acid, lysine, aspartic acid, ornithine, cysteine, homocysteine, or arginine; or Xaa14-Xaa17 is selected from the group consisting of: His-Lys-Lys-Lys (SEQ ID NO: 6), His-Leu-Lys-Lys (SEQ ID NO: 7), Lys-Lys-Lys-Lys (SEQ ID NO: 8), and His-Leu-Lys-Ser (SEQ ID NO: 9); and Y is, His-X, His-Asn-X, or His-Asn-Phe-X; where X is NH₂ or OH.

In another embodiment, the present invention is an antibody (one or more) or antigen binding fragment thereof which has binding specificity for a cyclic analog of human parathyroid hormone (hPTH), wherein the cyclic analog comprises an amino acid sequence: [Leu²⁷]cyclo(Glu²²-Lys²⁶)hPTH-(1-31) (SEQ ID NO: 10).

Further, the present invention provides a method of producing an antibody or antigen binding fragment thereof, which has binding specificity for a cyclic analog of human parathyroid hormone (hPTH), wherein the cyclic analog comprises an amino acid sequence: SEQ ID NO: 1. The method comprises administering an antigenic peptide comprising: SEQ ID NO: 1 to an animal, under conditions in which an antibody which has binding specificity for the cyclic analog of hPTH is produced in the animal, and isolating the antibody or antigen binding fragment thereof from the animal.

In another embodiment, the present invention is a method of producing an antibody or antigen binding fragment thereof, which has binding specificity for a cyclic analog of human parathyroid hormone (hPTH), wherein the cyclic analog comprises an amino acid sequence selected from the group consisting of: SEQ ID NO: 2 and SEQ ID NO: 3. The method comprising administering an antigenic peptide comprising: SEQ ID NO: 1 to an animal, under conditions in which an antibody which has binding specificity for the cyclic analog of hPTH is produced in the animal, and isolating the antibody or antigen binding fragment thereof from the animal.

In another embodiment the present invention is a method of producing an antibody or antigen binding fragment thereof, which has binding specificity for a cyclic analog of human parathyroid hormone (hPTH), wherein the cyclic analog comprises an amino acid sequence selected from the group consisting of: SEQ ID NO: 4, SEQ ID NO: 5, SEQ ID NO: 10, SEQ ID NO: 12, or SEQ ID NO: 13. The method comprising administering an antigenic peptide comprising: SEQ ID NO: 1 to an animal, under conditions in which an antibody which has binding specificity for the cyclic analog of hPTH is produced in the animal, and isolating the antibody or antigen binding fragment thereof from the animal.

In one embodiment, the antigenic peptide is coupled to a carrier. In another embodiment, the carrier is mariculture keyhole limpet hemocyanin (mcKLH). In a further embodiment, the antibody is a monoclonal antibody. In a still further embodiment, the antibody is a polyclonal antibody. In a still further embodiment, the present invention is an antibody or antigen binding fragment thereof produced by the method described above.

In another embodiment, the present invention is a method of producing an antibody or antigen binding fragment thereof, which has binding specificity for a cyclic analog of human parathyroid hormone (hPTH) wherein the cyclic analog comprises an amino acid sequence: [Leu²⁷]cyclo(Glu²²-Lys²⁶)hPTH-(1-31) (SEQ ID NO: 10). The method comprises administering an antigenic peptide comprising the amino acid sequence: Cys-Met-Glu-Arg-Val-Glu-Trp-Leu-Arg-Lys-Leu-Gin-Val-NH₂ (SEQ ID NO: 12), which is cyclized between Glu⁶ and Lys¹⁰ to an animal, under conditions in which an antibody to SEQ ID NO: 12 is produced in the animal, and isolating the antibody or antigen binding fragment thereof from the animal. In another embodiment, the present invention is an antibody or antigen binding fragment thereof produced by the method described above.

The present invention also encompasses a method for detecting a cyclic analog of human parathyroid hormone (hPTH) in a sample, wherein said cyclic analog comprises an amino acid sequence: SEQ ID NO: 1. The method comprises combining the sample with an antibody or antigen binding fragment thereof, which has binding specificity for the cyclic analog of HPTH, under conditions suitable for formation of an immunocomplex between the antibody and the cyclic analog of hPTH, and detecting the immunocomplex, wherein, detection of the immunocomplex indicates the presence of the cyclic analog of hPTH in the sample.

In another embodiment, the present invention is a method for detecting a cyclic analog of human parathyroid hormone (hPTH) in a sample, wherein said cyclic analog comprises an amino acid sequence: SEQ ID NO: 1. The method comprises combining the sample, a first antibody or antigen binding fragment thereof which has binding specificity for the cyclic analog of hPTH, and a second antibody which binds the cyclic analog of HPTH, under conditions in which the first antibody and the second antibody bind the cyclic analog of hPTH, thereby forming an immunocomplex, and detecting the immunocomplex, wherein, detection of the immunocomplex indicates the presence of the cyclic analog of hPTH in the sample.

In one embodiment, the second antibody binds a non-cyclic region of the cyclic analog of hPTH. In another embodiment the non-cyclic region is an N-terminal region of the cyclic analog of HPTH.

In another embodiment, the present invention is a method for detecting a cyclic analog of human parathyroid hormone (hPTH) in a sample, wherein said cyclic analog comprises an amino acid sequence selected from the group consisting of: SEQ ID NO: 2 and SEQ ID NO: 3. The method comprises combining the sample, a first antibody or antigen binding fragment thereof which has binding specificity for the cyclic analog of hPTH, and a second antibody which binds the cyclic analog of hPTH, under conditions in which the first antibody and the second antibody bind the cyclic analog of hPTH, thereby forming an immunocomplex, and detecting the immunocomplex, wherein, detection of the immunocomplex indicates the presence of the cyclic analog of HPTH in the sample. In this embodiment the second antibody binds a non-cyclic region of the cyclic analog of HPTH.

In another embodiment, the present invention is a method for detecting a cyclic analog of human parathyroid hormone (hPTH) in a sample, wherein said cyclic analog comprises an amino acid sequence: SEQ ID NO: 4. The method comprises combining the sample, a first antibody or antigen binding fragment thereof which has binding specificity for the cyclic analog of hPTH, and a second antibody which binds the cyclic analog of hPTH, under conditions in which the first antibody and the second antibody bind the cyclic analog of hPTH, thereby forming an immunocomplex, and detecting the immunocomplex, wherein, detection of the immunocomplex indicates the presence of the cyclic analog of hPTH in the sample. In this embodiment the second antibody binds a non-cyclic region of the cyclic analog of hPTH.

In another embodiment, the present invention is a method for detecting a cyclic analog of human parathyroid hormone (hPTH) in a sample, wherein said cyclic analog comprises an amino acid sequence: SEQ ID NO: 5. The method comprises combining the sample, a first antibody or antigen binding fragment thereof which has binding specificity for the cyclic analog of hPTH, and a second antibody which binds the cyclic analog of hPTH, under conditions in which the first antibody and the second antibody bind the cyclic analog of hPTH, thereby forming an immunocomplex, and detecting the immunocomplex, wherein, detection of the immunocomplex indicates the presence of the cyclic analog of hPTH in the sample.

In another embodiment, the present invention is a method for detecting a cyclic analog of human parathyroid hormone (hPTH) in a sample, wherein said cyclic analog comprises an amino acid sequence: [Leu²⁷]cyclo(Glu²²-Lys²⁶)hPTH-(1-31) (SEQ ID NO: 10). The method comprises combining the sample with an antibody or antigen binding fragment thereof, which has binding specificity for the cyclic analog of hPTH, under conditions suitable for formation of an immunocomplex between [Leu²⁷]cyclo(Glu²²-Lys²⁶)hPTH-(1-31) (SEQ ID NO: 10) and the antibody, and detecting the immunocomplex, wherein, detection of the immunocomplex indicates the presence of [Leu²⁷]cyclo(Glu²²-Lys²⁶)hPTH-(1-31) (SEQ ID NO: 10) in the sample.

In one embodiment, the first antibody is labeled with a horseradish peroxidase (HRP) enzymatic marker. In another embodiment, the second antibody is bound to biotin. In a further embodiment, the sample is combined with the first and second antibodies simultaneously. In a still further embodiment, the sample is combined with the first and second antibodies sequentially. In a still further embodiment, the sample is obtained from a subject being treated with the cyclic analog of hPTH.

In another embodiment, the present invention is a method for detecting a cyclic analog of human parathyroid hormone (hPTH) in a sample, wherein said cyclic analog comprises an amino acid sequence: [Leu²⁷]cyclo(Glu²²-Lys²⁶)hPTH-(1-31) (SEQ ID NO: 10). The method comprises combining the sample with an antibody or antigen binding fragment thereof, which has binding specificity for a cyclic analog of hPTH, under conditions suitable for formation of an immunocomplex between [Leu²⁷]cyclo(Glu²²-Lys²⁶)hPTH-(1-31) (SEQ ID NO: 10) and the antibody, and detecting the immunocomplex, wherein, detection of the immunocomplex indicates the presence of [Leu²⁷]cyclo(Glu²²-Lys²⁶)hPTH-(1-31) (SEQ ID NO: 10) in the sample.

In another embodiment, the present invention is a method for detecting a cyclic analog of human parathyroid hormone (HPTH) in a sample, wherein said cyclic analog comprises an amino acid sequence: [Leu²⁷]cyclo(Glu²²-Lys²⁶)hPTH-(1-31) (SEQ ID NO: 10). The present invention comprises combining the sample, a first antibody or antigen binding fragment thereof which has binding specificity for a cyclic analog of hPTH, and a second antibody which binds: [Leu²⁷]cyclo(Glu²²-Lys²⁶)hPTH-(1-31) (SEQ ID NO: 10), under conditions in which the first antibody and the second antibody bind to SEQ ID NO: 10, thereby forming an immunocomplex, and detecting the immunocomplex, wherein, detection of the immunocomplex indicates the presence of [Leu²⁷]cyclo(Glu²²-Lys²⁶)hPTH-(1-31) (SEQ ID NO: 10) in the sample.

In one embodiment, the first antibody is labeled with a horseradish peroxidase (HRP) enzymatic marker. In yet another embodiment, the second antibody is bound to biotin. In a further embodiment, the sample is combined with the first and second antibodies simultaneously. In a still further embodiment, the sample is combined with the first and second antibodies sequentially.

In another embodiment, the present invention is a method for detecting a cyclic analog of human parathyroid hormone (hPTH) in a sample, wherein said cyclic analog comprises an amino acid sequence SEQ ID NO: 1 The method comprises combining the sample, a first antibody or antigen binding fragment thereof which has binding specificity for the cyclic analog of hPTH, and a second antibody or antigen binding fragment thereof which has binding specificity for the cyclic analog of hPTH, under conditions in which the first antibody and the second antibody compete for an epitope on the cyclic analog of hPTH; forming an immunocomplex with the cyclic analog of hPTH with either the first or second antibody; and detecting of the immunocomplex; wherein, detection of the immunocomplex indicates the presence of the cyclic analog of HPTH in the sample.

Further, the present invention is a kit comprising an antibody or antigen binding fragment thereof which has binding specificity for a cyclic analog of human parathyroid hormone (hPTH), wherein the cyclic analog comprising an amino acid sequence: SEQ ID NO: 1.

In another embodiment, the present invention is a kit comprising an antibody or antigen binding fragment thereof which has binding specificity for a cyclic analog of human parathyroid hormone (hPTH), wherein the cyclic analog comprising an amino acid sequence selected from the group consisting of: SEQ ID NO: 2 and SEQ ID NO: 3.

In another embodiment, the present invention is a kit comprising an antibody or antigen binding fragment thereof which has binding specificity for a cyclic analog of human parathyroid hormone (hPTH), wherein the cyclic analog comprising an amino acid sequence selected from the group consisting of: SEQ ID NO: 4, SEQ ID NO: 5, or SEQ ID NO: 10. In yet another embodiment the antibody or antigen binding fragment thereof is coupled with a detectable label. In a still further embodiment, the kit further comprises a reagent for detecting said label; a second antibody, which binds a cyclic analog of hPTH; and washing buffers, diluents, solvents and stop solutions.

In another embodiment, the present invention is a kit comprising: an enzyme labeled first antibody or antigen binding fragment thereof which has binding specificity for a cyclic analog of hPTH; a biotinylated second antibody, which binds: [Leu²⁷]cyclo(Glu²²-Lys²⁶)hPTH-(1-31) (SEQ ID NO: 10); a color-producing substrate solution for use as a substrate for said enzyme; streptavidin coated microtiter plates; and washing buffers, diluents, solvents and stop solutions.

Further, the present invention is an antigenic peptide consisting of an amino acid sequence: Glu-Trp-Leu-Arg-Lys (SEQ ID NO: 1) which is cyclized between Glu¹ and Lys⁵.

In another embodiment, the present invention is an antigenic peptide consisting of an amino acid sequence: Glu-Trp-Leu-Arg-Lys-Leu-Leu (SEQ ID NO: 2) which is cyclized between Glu¹ and Lys⁵.

In another embodiment, the present invention is an antigenic peptide consisting of an amino acid sequence: Met-Glu-Arg-Val-Glu-Trp-Leu-Arg-Lys-Leu-Leu-Gln-Asp-Val (SEQ ID NO: 3) which is cyclized between Glu⁵ and Lys⁹.

In another embodiment, the present invention is an antigenic peptide consisting of an amino acid sequence: Cys-Met-Glu-Arg-Val-Glu-Trp-Leu-Arg-Lys-Leu-Leu-Gln-Asp-Val-NH₂ (SEQ ID NO: 12) which is cyclized between Glu⁶ and Lys¹⁰.

The antibodies and methods of the invention have the particular advantage of providing binding specificity for cyclic analogs of hPTH. In particular, the antibodies and methods are designed to provide novel recognition for the cyclic analogs of hPTH due to the negligible or absence of cross-reactivity that the antibodies have with linear hPTH analogs.

### BRIEF DESCRIPTION OF THE DRAWINGS

FIG. 1 is a structural representation of [Leu²⁷]cyclo(Glu²²-Lys²⁶)hPTH-(11-31).

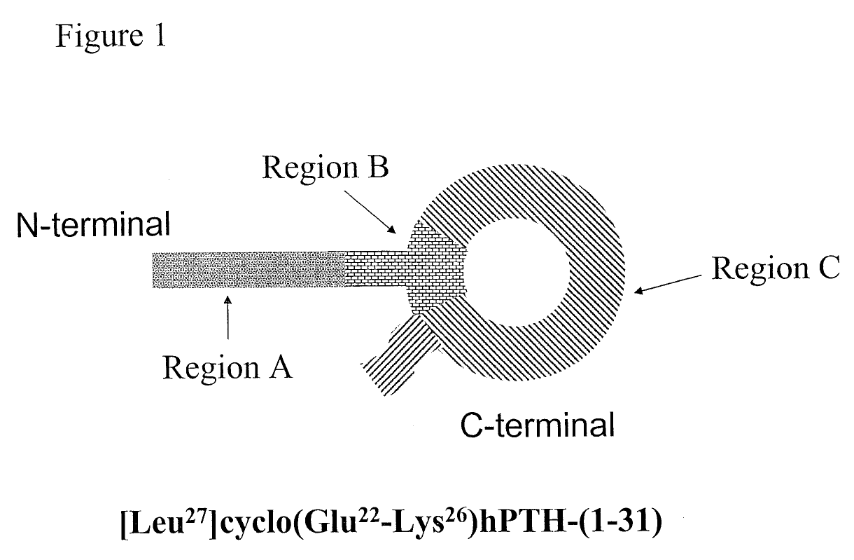

FIG. 2 is a standard curve for [Leu²⁷]cyclo(Glu²²-Lys²⁶)hPTH-(1-31) obtained using a two-site sandwich ELISA as described in Example 5.

FIG. 3 is a bar graph showing that the antibodies which have binding specificity for cyclic analogs of hPTH, do not cross react with non-cyclic analogs of hPTH as described in Example 6.

### DETAILED DESCRIPTION OF THE INVENTION

The present invention relates to antibodies or antigen binding fragments thereof, which have binding specificity for cyclic analogs of hPTH, and methods for utilizing such antibodies.

Human parathyroid hormone (hPTH) is a polypeptide consisting of 84 amino acids represented by the following amino acid sequence:

| | |
|---|---|
| 10 | |
| Ser-Val-Ser-Glu-Ile-Gln-Leu-Met-His-Asn-Leu-Gly-Lys-His-Leu-Asn-Ser-Met-Glu- | (SEQ ID NO:11) |
| 20 30 | |
| Arg-Val-Glu-Trp-Leu-Arg-Lys-Lys-Leu-Gln-Asp-Val-His-Asn-Phe-Val-Ala-Leu-Gly- | |
| 40 50 | |
| Ala-Pro-Leu-Ala-Pro-Arg-Asp-Ala-Gly-Ser-Gln-Arg-Pro-Arg-Lys-Lys-Glu-Asp-Asn- | |
| 60 70 | |
| Val-Leu-Val-Glu-Ser-His-Glu-Lys-Ser-Leu-Gly-Glu-Ala-Asp-Lys-Ala-Asp-Val-Asn- | |
| 80 | |
| Val-Leu-Thr-Lys-Ala-Lys-Ser-Gln-OH. | |

hPTH is a hypercalcemic hormone, which functions to elevate blood calcium levels by increasing resorption of bone calcium, increasing absorption of calcium from the intestine, and increasing renal absorption of calcium from nascent urine in the kidney tubules. Although continuously infused low levels of HPTH can remove calcium from bone, the same low doses when intermittently injected can actually promote bone growth. Analogs of hPTH have been found to have increased activities in bone restoration.

As used herein, the phrase “analog of hPTH” encompasses any natural or synthetic polypeptide having an amino acid sequence that is similar or substantially similar to hPTH. For example, an analog of hPTH can share about 25%, 35%, 50%, 60%, 70%, 80%, 85%, 90%, 95%, or 99% identity to the amino acid sequence of hPTH. An analog of hPTH includes those having amino acid substitutions, deletions and/or additions. For example, the analog can have one or more conservative amino acid substitutions where in one or more amino acids are replaced by an amino acid(s) that has similar chemical and/or physical properties (e.g., charge, structure, polarity, hydrophobicity, hydrophilicity). Replacement of an amino acid by another within the following groups is a conservative amino acid substitution; Ala, Val, Leu, Ile; Ser, Thr; Asp, Glu; Asn, Gln; Lys, Arg; Phe, Tyr. In other embodiments, one or more amino acid substitutions can be made to hPTH which enhance the biological activities of hPTH. For example, U.S. Pat. No. 5,955,425 describes HPTH analogs in which Lys²⁷ is substituted by hydrophobic residues such as Leu, Ile, norleucine, Met, Val, Ala, Trp, or Phe.

In addition analogs of hPTH encompass natural or synthetic peptides which possess at least one biological activity of hPTH. Biological activities of HPTH include the ability to elevate blood calcium levels by increasing resorption of bone calcium, increasing absorption of calcium from the intestine, and increasing renal absorption of calcium from nascent urine in the kidney tubules.

Analogs of HPTH also include fragments where the naturally occurring amino acids of hPTH (SEQ ID NO: 11) are deleted. For example, hPTH analogs include amino acids 1-34, amino acids 1-33, amino acids 1-32, amino acids 1-31, amino acids 1-29, and amino acids 1-28 of hPTH SEQ ID NO: 11.

As used herein, the phrase “cyclic analog of hPTH” encompasses any natural or synthetic HPTH polypeptide, as described above, which is cyclized between at least one amino acid pair of hPTH (between at least two amino acids of hpTH). As used herein the term “cyclized” includes peptide chains in which at least one pair of amino acids are linked together to form a cyclic region on the chain. As used herein, the term “cyclic region” incorporates all amino acids between, and including two joined, non-adjacent, amino acids in a peptide chain. For example, in the cyclic hPTH analog [Leu²⁷] cyclo (Glu²²-Lys²⁶) hPTH-(1-31) (SEQ ID NO: 10), the cyclic region incorporates amino acids 22, 23, 34, 25 and 26. In a particular embodiment, the HPTH analog is cyclized by the formation of a lactam, involving the coupling of the side-chains of selected amino acid pairs such as between amino acids Glu²² and Lys²⁶, or Lys²⁶ and Asp³⁰ of hPTH SEQ ID NO: 11 and fragments thereof. Other types of cyclizations are also possible such as those containing a thioester, ester or ether, or, for example, a cyclic analog of hPTH can be formed by the formation of a disulfide bridge at amino acids Lys¹³ and Ser¹⁷ of HPTH SEQ ID NO: 11 when both positions are substituted with cysteine residues. Cyclizations at other positions are also encompassed by the invention.

Various analogs of hPTH which are active in the restoration of bone loss are disclosed in U.S. Pat. No. 5,556,940, U.S. Pat. No. 5,955,425, U.S. Pat. No. 6,110,892, U.S. Pat. No. 6,316,410, and U.S. Pat. No. 6,541,450. The entire contents of each U.S. patent listed above are incorporated herein by reference.

In one embodiment the cyclic analog of hPTH comprises the amino acid sequence: Glu-Trp-Leu-Arg-Lys (SEQ ID NO: 1) wherein cyclization occurs between Glu¹ and Lys⁵.

In another embodiment the cyclic analog of HPTH comprises the amino acid sequence: Glu-Trp-Leu-Arg-Lys-Leu-Leu (SEQ ID NO: 2) wherein cyclization occurs between Glu¹ and Lys⁵.

In another embodiment the cyclic analog of hPTH is represented by the amino acid sequence: Met-Glu-Arg-Val-Glu-Trp-Leu-Arg-Lys-Leu-Leu-Gln-Asp-Val (SEQ ID NO: 3) wherein cyclization occurs between Glu⁵ and Lys⁹.

In another embodiment the cyclic analog of hPTH is represented by the amino acid sequence:

| | |
|---|---|
| 10 | |
| R-NH-Xaa1-Val-Ser-Glu-Ile-Gln-Leu-Xaa8-His-Asn-Leu-Gly-Xaa13-Xaa14-Xaa15- | (SEQ ID NO:4) |
| 20 30 | |
| Xaa16-Xaa17-Met-Glu-Arg-Val-Glu-Trp-Leu-Arg-Lys-Leu-Leu-Gln-Asp-Val-Y | |

wherein: the cyclic analog is cyclized between Glu²² and Lys²⁶; R is a hydrogen or any linear or branched chain alkyl, acyl or aryl group; Xaa1 is serine, alanine, norleucine, or a-aminoisobutyric acid; Xaa8 is methionine, norisoleucine, or a hydrophobic amino acid; Xaa13 is lysine, ornithine, glutamic acid, aspartic acid, cysteine, or homocysteine; Xaa14 is histidine or a water soluble amino acid; Xaa15 is leucine or a water soluble amino acid; Xaa16 is asparagine or a water soluble amino acid; Xaa17 is serine or a water soluble amino acid; and Y is, His-X, His-Asn-X, or His-Asn-Phe-X; where X is NH₂ or OH.

In another embodiment the cyclic analog of hPTH is represented by the amino acid sequence:

| | |
|---|---|
| 10 | |
| H-NH-Ser-Val-Ser-Glu-Ile-Gln-Leu-Met-His-Asn-Leu-Gly-Xaa13-Xaa14-Xaa15- | (SEQ ID NO:5) |
| 20 30 | |
| Xaa16-Xaa17-Met-Glu-Arg-Val-Glu-Trp-Leu-Arg-Lys-Leu-Leu-Gln-Asp-Val-Y | |

wherein: the cyclic analog is cyclized between Glu²² and Lys²⁶; Xaa14 is histidine or lysine; Xaa15 is leucine, lysine, or arginine; Xaa16 is asparagine, ornithine, homocitrulline, aspartic acid, arginine, lysine, d-lysine, serine, or glycine; Xaa17 is serine, glutamic acid, lysine, aspartic acid, ornithine, cysteine, homocysteine, or arginine; and Y is, His-X, His-Asn-X, or His-Asn-Phe-X; where X is NH₂ or OH. In another embodiment the amino acid sequence of Xaa14-Xaa17 is selected from the group consisting of: His-Lys-Lys-Lys (SEQ ID NO: 6), His-Leu-Lys-Lys (SEQ ID NO: 7), Lys-Lys-Lys-Lys (SEQ ID NO: 8), and His-Leu-Lys-Ser (SEQ ID NO: 9).

In a particular embodiment the cyclic analog of hPTH is represented by the amino acid sequence SEQ ID NO: 10:

| | |
|---|---|
| 10 | |
| H-NH-Ser-Val-Ser-Glu-Ile-Gln-Leu-Met-His-Asn-Leu-Gly-Lys-His-Leu-Asn-Ser | (SEQ ID NO:10) |
| 20 30 | |
| Met-Glu-Arg-Val-Glu-Trp-Leu-Arg-Lys-Leu-Leu-Gln-Asp-Val | |

wherein: the cyclic analog is cyclized between Glu²² and Lys²⁶.

As used herein an “alkyl group” is a saturated hydrocarbon in a molecule that is bonded to one other group in the molecule through a single covalent bond from one of its carbon atoms. Alkyl groups can be cyclic or acyclic, branched or unbranched (straight chained) and substituted or unsubstituted when straight chained or branched. An alkyl group typically has from about one to about twelve carbon atoms, for example, about one to about six carbon atoms, or about one to about four carbon atoms. When cyclic, an alkyl group typically contains from about 3 to about 10 carbons, for example, from about 3 to about 8 carbon atoms, e.g., a cyclopropyl group, a cyclobutyl group, a cyclopentyl group, a cyclohexyl group, a cycloheptyl group or a cyclooctyl group.

As used herein “acyl groups” are represented by the formula —C(O)R, where R is an alkyl group. One or more of the hydrogen atoms of an acyl group can be substituted. Suitable substituents for alkyl and acyl groups include —OH, —O(R′), —O—CO—(R′), —NO₂, —COOH, ═O, —NH₂, —NH(R′), —N(R′)₂, —COO(R′), —CONH₂, —CONH(R′), —CON(R′)₂, and guanidine. Each R′ is independently an alkyl group or an aryl group. These groups can additionally be substituted by an aryl group (e.g., an alkyl group can be substituted with an aromatic group to form an arylalkyl group). A substituted alkyl or acyl group can have more than one substituent.

As used herein, “aryl groups” include carbocyclic aromatic groups such as phenyl, p-tolyl, 1-naphthyl, 2-naphthyl, 1-anthracyl and 2-anthracyl. Aryl groups also include heteroaromatic groups such as N-imidazolyl, 2-imidazole, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl.

Aryl groups also include fused polycyclic aromatic ring systems in which a carbocyclic, alicyclic, or aromatic ring or heteroaryl ring is fused to one or more other heteroaryl or aryl rings. Examples include 2-benzothienyl, 3-benzothienyl, 2-benzofuranyl, 3-benzofuranyl, 2-indolyl, 3-indolyl, 2-quinolinyl, 3-quinolinyl, 2-benzothiazole, 2-benzooxazole, 2-benzimidazole, 2-quinolinyl, 3-quinolinyl, 1-isoquinolinyl, 3-quinolinyl, 1-isoindolyl and 3-isoindolyl.

Suitable naturally occurring “hydrophobic amino acids” of the present invention, include but are not limited to, leucine, isoleucine, alanine, valine, phenylalanine, proline, methionine, and glycine. Combinations of hydrophobic amino acids can also be employed.

Suitable naturally occurring “water soluble amino acids” include, but are not limited to, serine, histidine, asparagine, aspartate and glutamate, lysine, arginine, glutamine, cysteine, threonine, ornithine, and tyrosine. Combinations of water soluble amino acids can also be employed.

Non-naturally occurring amino acids can also be employed which include, for example, beta-amino acids. Both D and L configurations and racemic mixtures of amino acids can be employed. Suitable amino acids can also include amino acid derivatives or analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C₁-C₈ hydrocarbons which are completely saturated, which contain one or two heteroatoms such as nitrogen, oxygen or sulfur and/or which contain one or more units of unsaturation. Aromatic or aryl groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F), —O (aliphatic, substituted aliphatic, benzyl, substituted benzyl, aryl or substituted aryl group), —CN, —NO₂, —COOH, —NH₂, —NH (aliphatic group, substituted aliphatic, benzyl, substituted benzyl, aryl or substituted aryl group), —N (aliphatic group, substituted aliphatic, benzyl, substituted benzyl, aryl or substituted aryl group)₂, —COO (aliphatic group, substituted aliphatic, benzyl, substituted benzyl, aryl or substituted aryl group), —CONH₂, —CONH (aliphatic, substituted aliphatic group, benzyl, substituted benzyl, aryl or substituted aryl group), —SH, —S (aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lipophilicity or hydrophobicity of natural amino acids which are hydrophilic.

A number of the suitable amino acids, amino acid analogs and salts thereof can be obtained commercially. Others can be synthesized by methods known in the art. Synthetic techniques are described, for example, in Green and Wuts, “Protecting Groups in Organic Synthesis”, John Wiley and Sons, Chapters 5 and 7, 1991.

Also included in the present invention are pharmaceutically acceptable salts of the cyclic analogs of hPTH described herein. Cyclic analogs of HPTH disclosed herein which possess a sufficiently acidic, a sufficiently basic functional groups or both, can react with any of a number of organic or inorganic base, and inorganic and organic acids, to form a salt.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like. Base addition salts also include those derived from organic bases such as methylamine, ethylamine, and triethylamine.

Acids commonly employed to form acid addition salts from cyclic analogs of hPTH with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

In one embodiment, the present invention is an antibody (one or more) or antigen binding fragment thereof, which has binding specificity for a cyclic analog of human parathyroid hormone (hPTH). As used herein, the term “antibody” encompasses both polyclonal and monoclonal antibodies. The terms polyclonal and monoclonal refer to the degree of homogeneity of an antibody preparation, and are not intended to be limited to particular methods of production. Polyclonal antibodies are heterogeneous populations of antibody molecules derived from the sera of animals immunized with an antigen. A monoclonal antibody contains a substantially homogeneous population of antibodies specific to antigens, which population contains substantially similar epitope binding sites. Such antibodies may be of any immunoglobulin class including IgG, IgM, IgE, IgA, IgD, and any subclass thereof. The antibody or antigen binding fragment thereof can be isolated or purified. As used herein “isolated” or “purified” (e.g., partially or substantially) encompasses an antibody of antigen binding fragment thereof which has been separated away from other material, such as the materials contained in the medium in which it was produced. In one embodiment, the isolated or purified antibody or antigen binding fragment is part of a composition (crude extract). In another embodiment, the antibody or antigen binding fragment is substantially free of materials or contaminating proteins from the source from which the antibody or antigen binding fragment is derived, or substantially free from chemical precursors or other chemicals when recombinantly produced.

Antibodies or antigen-binding fragments thereof which have binding specificity for a cyclic analog of hPTH include, for example, single chain antibodies, chimeric antibodies, mammalian (e.g., human) antibodies, humanized antibodies, CDR-grafted antibodies (e.g., primatized antibodies), veneered antibodies, multivalent antibodies (e.g., bivalent) and bispecific antibodies. Chimeric, CDR-grafted or veneered single chain antibodies, comprising portions derived from different species, are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

As used herein, the phrase “mammalian antibody” includes an antibody in which the variable and constant regions (if present) have amino acid sequences that are encoded by nucleotide sequences derived from mammalian germline immunoglobulin genes. A “mammalian antibody” can include sequences that are not encoded in the germline (e.g., due to N nucleotides, P nucleotides, and mutations that can occur as part of the processes that produce high-affinity antibodies such as, somatic mutation, affinity maturation, clonal selection) that occur as a result of biological processes in a suitable in vivo expression system (e.g., a human, a transgenic animal expressing a human antibody). In one embodiment, the antibody is a human antibody in which the variable and constant regions (if present) have amino acid sequences that are encoded by nucleotide sequences derived from human (Homo sapiens) germline immunoglobulin genes. Antibodies, antigen-binding fragments thereof and portions or regions of antibodies can be produced, for example, by expression of a nucleic acid of non-human origin (e.g., a synthetic nucleic acid) that has the requisite nucleotide sequence.

As used herein, the term “CDR-grafted antibody” includes an antibody that comprises a complementarity-determining region (CDR) that is not naturally associated with the framework regions of the antibody. Generally the CDR is from an antibody from a first species and the framework regions and constant regions (if present) are from an antibody from a different species. The CDR-grafted antibody can be a “humanized antibody”.

As used herein, the term “humanized antibody” includes an antibody comprising a CDR that is not of human origin and framework and/or constant regions that are of human origin. For example, a humanized antibody can comprise a CDR derived from an antibody of nonhuman origin (e.g., natural antibody such as a murine (e.g., mouse, rat) antibody, artificial antibody), and framework and constant regions (if present) of human origin (e.g., a human framework region, a human consensus framework region, a human constant region (e.g., C_{L}, C_{H}1, hinge, C_{H}2, C_{H}3, C_{H}4)). CDR-grafted single chain antibodies containing a CDR of non-human origin and framework and constant regions (if present) of human origin (e.g., CDR-grafted scFv) are also encompassed by the term humanized antibody.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213).

As used herein, the term “chimeric antibody” includes an antibody comprising portions of immunoglobulins from different origin. None of the portions of immunoglobulins that comprise a chimeric antibody need to be of human origin. For example, a chimeric antibody can comprise an antigen-binding region of nonhuman region (e.g., rodent) and a constant region of non-human primate origin (e.g., a chimpanzee framework region, a chimpanzee constant region (e.g., C_{L}, C_{H}1, hinge, C_{H}2, C_{H}3, C_{H}4)).

Antibodies of the invention can be single chain antibodies (e.g., a single chain Fv (scFv)) and can include a linker moiety (e.g., a linker peptide) not found in native antibodies. For example, an scFv can comprise a linker peptide, such as about two to about twenty glycine residues or other suitable linker, which connects a heavy chain variable region to a light chain variable region.

In addition, antibodies of the invention can be bispecific antibodies. As used herein, a “bispecific antibody” includes an antibody that binds two different types of antigen. Bispecific antibodies can be secreted by triomas and hybrid hybridomas. Generally, triomas are formed by fusion of a hybridoma and a lymphocyte (e.g., antibody secreting B cell) and hybrid hybridomas are formed by fusion of two hybridomas. Each of the cells that are fused to produce a trioma or hybrid hybridoma produces a monospecific antibody. However, triomas and hybrid hybridomas can produce an antibody containing antigen binding sites which recognize different antigens. The supernatants of triomas and hybrid hybridomas can be assayed for bispecific antibody using a suitable assay (e.g., ELISA), and bispecific antibodies can be purified using conventional methods (see, e.g., U.S. Pat. No. 5,959,084 (Ring et al.), U.S. Pat. No. 5,141,736 (Iwasa et al.), U.S. Pat. Nos. 4,444,878, 5,292,668 and 5,523,210 (Paulus et al.) and U.S. Pat. No. 5,496,549 (Yamazaki et al.)).

Antigen-binding fragments encompass functional fragments of antibodies including, e.g., fragments of single chain antibodies, chimeric antibodies, human antibodies, humanized antibodies, CDR-grafted antibodies (e.g., primatized antibodies), veneered antibodies and bispecific antibodies. Antigen-binding fragments further encompass Fv, Fab, Fab′ and F(ab′)₂ fragments. Antigen-binding fragments, such as Fv, Fab, Fab′ and F(ab′)₂ fragments, can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antigen-binding fragments can also be produced recombinantly using antibody genes in which one or more stop codons have been introduced upstream of the natural stop site. For example, a chimeric gene encoding a F(ab′)₂ heavy chain portion can be designed to include DNA sequences encoding the C_{H}1 domain and hinge region of the heavy chain.

In addition the antibodies of the present invention can be fusion proteins or immunoconjugates in which the antibody moiety (e.g., antibody or antigen-binding fragment thereof, antibody chain or antigen-binding portion thereof) is linked directly or indirectly to a non-immunoglobulin moiety (i.e., a moiety which does not occur in immunoglobulins as found in nature). Fusion proteins comprise an antibody moiety and a non-immunoglobulin moiety that are components of a single continuous polypeptide chain. The non-immunoglobulin moiety can be located N-terminally, C-terminally or internally with respect to the antibody moiety.

In other embodiments, the antibody moiety and non-immunoglobulin moiety may not be part of a continuous polypeptide chain, but can be connected or conjugated directly or indirectly through any suitable linker. Suitable methods for connecting or conjugating the moieties are well known in the art. (See, e.g., Ghetie et al., Pharmacol. Ther. 63:209-34 (1994)). A variety of suitable linkers (e.g., heterobifunctional reagents) and methods for preparing immuno-conjugates are well known in the art. (See, for example, Hermanson, G. T., Bioconjugate Techniques, Academic Press: San Diego, Calif. (1996).) Suitable non-immunoglobulin moieties for inclusion in an immuno-conjugate include a therapeutic moiety such as a toxin (e.g., cytotoxin, cytotoxic agent), a therapeutic agent (e.g., a chemotherapeutic agent, an antimetabolite, an alkylating agent, an anthracycline, an antibiotic, an anti-mitotic agent, a biological response modifier (e.g., a cytokine (e.g., an interleukin, an interferon, a tumor necrosis factor), a growth factor (e.g., a neurotrophic factor)), a plasminogen activator), a radionuclide (e.g, a radioactive ion), an enzyme and the like.

As used herein, the terms “antigen” or “antigenic peptide” encompass a molecule or portion of a molecule capable of being bound by an antibody, or antigen binding fragment thereof, which is additionally capable of inducing an animal to produce an antibody capable of binding to an epitope of that antigen. The antigen reacts, in a highly selective manner, with its corresponding antibody and not with the multitude of other antibodies which may be evoked by other antigens. In a particular embodiment the antigen or antigenic peptide is a cyclic analog of hPTH.

As used herein, the term “epitope” refers to that portion of an antigen or antigenic peptide capable of being recognized and bound by an antibody or antigen binding fragment thereof. An antigen or antigenic peptide may contain more than one epitope. In one embodiment, the antibodies or antigen binding fragments thereof, of the present invention, will bind to an epitope on a cyclic analog of hPTH, wherein the epitope is the cyclic region of the cyclic analog of hPTH. In another embodiment, the antibodies or antigen binding fragments thereof of the present invention will bind to an epitope on a cyclic analog of hPTH, wherein the epitope comprises at least one amino acid from the cyclic region of the cyclic analog of hPTH.

As used herein, an antibody or antigen binding fragment thereof, of the present invention, has “binding specificity” for a cyclic analog of hPTH if it binds to the cyclic analog of hPTH with greater affinity than it binds to a non-cyclic analog of hPTH. If the antibody binds to a cyclic analog of hPTH with greater affinity than it binds to a non-cyclic analog of hPTH, the antibody will bind, at least in part, to the cyclic region of the hPTH cyclic analog (the antibody binds to all or a portion of the cyclic region of the hPTH cyclic analog). In a particular embodiment, an antibody or antigen binding fragment thereof of the present invention which has binding specificity for a cyclic analog of HPTH will recognize and bind at least one amino acid of SEQ ID NO: 1.

As used herein, an antibody or antigen binding fragment thereof, of the present invention, has binding specificity for a cyclic analog of hPTH if it binds to the cyclic analog of hPTH with at least 20% greater affinity, at least 50% greater affinity, at least 80% greater affinity, or at least 90% greater affinity, than it binds to a non-cyclic analog of hPTH.

The invention is directed to an antibody (one or more) or antigen binding fragment thereof, which has binding specificity for a cyclic analog of hPTH. The cyclic analog may be an isolated peptide chain, or may represent the cyclic region of a larger peptide. In one embodiment, the invention is directed to an antibody or antigen binding fragment thereof which has binding specificity for a cyclic analog of hPTH, wherein the cyclic analog comprises an amino acid sequence: Glu-Trp-Leu-Arg-Lys (SEQ ID NO: 1). SEQ ID NO: 1 is cyclized between amino acids at positions Glu¹ and Lys⁵. SEQ ID NO: 1 may be an isolated peptide chain, or may represent the cyclic region of a larger peptide chain. In a particular embodiment SEQ ID NO: 1 represents a cyclic region of a bioactive hPTH cyclic analog. As used herein, the term “bioactive hPTH cyclic analog”, refers to any natural or synthetic hPTH analog, as described above, that has a cyclic region and at least one biological activity of hPTH. In a particular embodiment SEQ ID NO: 1 represents positions 22 to 26 of [Leu²⁷] cyclo(Glu²²-Lys²⁶)hPTH-(1-31) (SEQ ID NO: 10). In a particular embodiment, the epitope recognized by an antibody or antigen binding fragment thereof which has binding specificity for a cyclic analog of hPTH, comprises at least one amino acid from positions 1 through 5 of SEQ ID NO: 1.

In another embodiment, the antibody or antigen binding fragment thereof has binding specificity for a cyclic analog of hPTH comprising the amino acid sequence: Glu-Trp-Leu-Arg-Lys-Leu-Leu (SEQ ID NO: 2). SEQ ID NO: 2 is cyclized between amino acids at positions Glu¹ and Lys⁵. SEQ ID NO: 2 may be an isolated peptide chain, or may represent a cyclic region of a larger peptide chain. In a particular embodiment SEQ ID NO: 2 represents amino acids from positions 22 to 28 of SEQ ID NO: 10. In a particular embodiment, the epitope recognized by an antibody or antigen binding fragment thereof which has binding specificity for a cyclic analog of hPTH, comprises at least one amino acid from positions 1 through 5 of SEQ ID NO: 2.

In another embodiment, the antibody or antigen binding fragment thereof has binding specificity for a cyclic analog of hPTH comprising the amino acid sequence: Met-Glu-Arg-Val-Glu-Trp-Leu-Arg-Lys-Leu-Leu-Gln-Asp-Val (SEQ ID NO: 3). SEQ ID NO: 3 is cyclized between amino acids at positions Glu⁵ and Lys⁹. SEQ ID NO: 3 may be an isolated peptide chain, or may represent a cyclic region of a larger peptide chain. In a particular embodiment SEQ ID NO: 3 represents amino acids from positions 18 to 31 of the cyclic HPTH analog SEQ ID NO: 10. In a particular embodiment, the epitope recognized by an antibody or antigen binding fragment thereof which has binding specificity for a cyclic analog of HPTH, comprises at least one amino acid from positions 5 through 9 of SEQ ID NO: 3.

In another embodiment, the antibody or antigen binding fragment thereof, has binding specificity for a cyclic analog of HPTH comprising the amino acid sequence:

| | |
|---|---|
| 10 | |
| R-NH-Xaa1-Val-Ser-Glu-Ile-Gln-Leu-Xaa8-His-Asn-Leu-Gly-Xaa13-Xaa14-Xaa15- | (SEQ ID NO:4) |
| 20 30 | |
| Xaa16-Xaa17-Met-Glu-Arg-Val-Glu-Trp-Leu-Arg-Lys-Leu-Leu-Gln-Asp-Val-Y | |

wherein: the cyclic analog is cyclized between Glu²² and Lys²⁶; R is a hydrogen or any linear or branched chain alkyl, acyl or aryl group; Xaa1 is serine, alanine, norleucine, or a-aminoisobutyric acid; Xaa8 is methionine, norisoleucine, or a hydrophobic amino acid; Xaa13 is lysine, ornithine, glutamic acid, aspartic acid, cysteine, or homocysteine; Xaa14 is histidine or a water soluble amino acid; Xaa15 is leucine or a water soluble amino acid; Xaa16 is asparagine or a water soluble amino acid; Xaa17 is serine or a water soluble amino acid; and Y is, His-X, His-Asn-X, or His-Asn-Phe-X; where X is NH₂ or OH. In a particular embodiment, the epitope recognized by an antibody or antigen binding fragment thereof which has binding specificity for a cyclic analog of hPTH, comprises at least one amino acid from positions 22 through 26 of SEQ ID NO: 4.

In another embodiment, the antibody or antigen binding fragment thereof, has binding specificity for a cyclic analog of hPTH comprising the amino acid sequence:

| | |
|---|---|
| 10 | |
| H-NH-Ser-Val-Ser-Glu-Ile-Gln-Leu-Met-His-Asn-Leu-Gly-Xaa13-Xaa14-Xaa15- | (SEQ ID NO:5) |
| 20 30 | |
| Xaa16-Xaa17-Met-Glu-Arg-Val-Glu-Trp-Leu-Arg-Lys-Leu-Leu-Gln-Asp-Val-Y | |

wherein: the cyclic analog is cyclized between Glu²² and Lys²⁶; Xaa14 is histidine or lysine; Xaa15 is leucine, lysine, or arginine; Xaa16 is asparagine, ornithine, homocitrulline, aspartic acid, arginine, lysine, d-lysine, serine, or glycine; Xaa17 is serine, glutamic acid, lysine, aspartic acid, ornithine, cysteine, homocysteine, or arginine; and Y is, His-X, His-Asn-X, or His-Asn-Phe-X; where X is NH₂ or OH. In another embodiment the amino acid sequence of Xaa14-Xaa17 is selected from the group consisting of: His-Lys-Lys-Lys (SEQ ID NO: 6), His-Leu-Lys-Lys (SEQ ID NO: 7), Lys-Lys-Lys-Lys (SEQ ID NO: 8), and His-Leu-Lys-Ser (SEQ ID NO: 9). In a particular embodiment, the epitope recognized by an antibody or antigen binding fragment thereof which has binding specificity for a cyclic analog of hPTH, comprises at least one amino acid from positions 22 through 26 of SEQ ID NO: 5.

In a particular embodiment, the invention is an antibody or antigen binding fragment thereof, which has binding specificity for a cyclic analog of hPTH comprising the amino acid sequence:

| | |
|---|---|
| 10 | |
| H-NH-Ser-Val-Ser-Glu-Ile-Gln-Leu-Met-His-Asn-Leu-Gly-Lys-His-Leu-Asn-Ser | (SEQ ID NO:10) |
| 20 30 | |
| Met-Glu-Arg-Val-Glu-Trp-Leu-Arg-Lys-Leu-Leu-Gln-Asp-Val | |

wherein: the cyclic analog is cyclized between Glu²² and Lys²⁶. In a particular embodiment, the epitope recognized by an antibody or antigen binding fragment thereof which has binding specificity for a cyclic analog of hPTH, comprises at least one amino acid from positions 22 through 26 of SEQ ID NO: 10.

The present invention is also directed to methods of producing an antibody (one or more) or antigen binding fragment thereof which has binding specificity for a cyclic analog of hPTH. The antibody can be produced using techniques known to those of skill in the art. For example, a variety of methods for preparing and using an antigenic peptide, and for producing polyclonal and monoclonal antibodies are known in the art (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)).

In one embodiment the methods of producing antibodies and antigen binding fragments thereof of the present invention comprises administering a cyclic analog of hPTH or fragments thereof to an animal under conditions in which an antibody to the cyclic analog of hPTH is produced in the animal. In one embodiment the antigenic peptide is SEQ ID NO: 1. In another embodiment the antigenic peptide is SEQ ID NO: 2. In yet another embodiment the antigenic peptide is SEQ ID NO: 3. In other embodiments the antigenic peptide is selected from the group consisting: SEQ ID NO: 4, SEQ ID NO: 5, SEQ ID NO: 10, SEQ ID NO: 12 and SEQ ID NO: 13.

A variety of animals (e.g., a rat, a mouse, a rabbit, a goat, a camel, a llama, a sheep, a chicken, or a human) can be used in the methods of production of an antibody or antigen binding fragment thereof. The administration of such antigenic peptides to the animal may be accomplished by any of a variety of methods, including but not limited to subcutaneous, intraperitoneal, or intramuscular injection. As will be appreciated the dose of antigenic peptide administered will correspondingly vary with the specific peptide utilized as well as the animal host. After the initial administration or immunization of the cyclic analog of hPTH, the animal may receive one or more additional immunization boosts.

Stimulators of the immune response in the animal, such as adjuvants, may also be administered in combination with the antibodies of the present invention. Examples of such adjuvants include, Freund's complete adjuvant, Freund's incomplete adjuvant, Montanide ISA adjuvant, Ribj's adjuvant, Hunter's TiterMax, and aluminium salt adjuvants.

The antibody titer of an animal that has been administered cyclic analog of hPTH can be monitored by any of a variety of techniques well-known in the art, such as routine bleed and the like. The antisera is then isolated (e.g., via centrifugation) and thereafter screened for the presence of antibodies which have binding affinities for the cyclic analogs of hPTH.

When approximately high titers of antibody are obtained, the antibody is isolated from the animal by collecting blood from the animal and recovering the antisera. The resultant antisera may be affinity purified to derive the antibodies of the present invention. As is well-known in the art, the antisera may be purified via conventional techniques, such as the introduction into a separation column with the aforementioned antigenic peptides bound to a solid phase. The antisera may then be washed to remove antibodies not having specificity for the antigenic peptides, with the remaining bound antibody specific for the antigenic peptides ultimately being eluted therefrom.

Generally for monoclonal antibodies a hybridoma is produced by fusing a suitable immortal cell line (e.g., a myeloma cell line or a heteromyeloma) with antibody-producing cells. Antibody producing cells can be produced from the peripheral blood or, preferably the spleen or lymph nodes, of suitable animals immunized with the antigen of interest. The fused cells (hybridomas) can be isolated using selective culture conditions, and cloned by limiting dilutions.

The present invention also relates to methods of producing antibodies using isolated and/or recombinant (including, e.g., essentially pure) nucleic acids comprising sequences which encode an antibody or antigen binding fragment (e.g., a human, humanized, chimeric antibody or light or heavy chain of any of the foregoing) or fusion protein of the invention.

Nucleic acids referred to herein as “isolated” are nucleic acids which have been separated away from other material (e.g., other nucleic acids such as genomic DNA, cDNA and/or RNA) in its original environment (e.g., in cells or in a mixture of nucleic acids such as a library). An isolated nucleic acid can be isolated as part of a vector (e.g., a plasmid). Nucleic acids can be naturally occurring, produced by chemical synthesis, by combinations of biological and chemical methods (e.g., semisynthetic), and be isolated using any suitable methods.

Nucleic acids referred to herein as “recombinant” are nucleic acids which have been produced by recombinant DNA methodology, including methods which rely upon artificial recombination, such as cloning into a vector or chromosome using, for example, restriction enzymes, homologous recombination, viruses and the like, and nucleic acids prepared using the polymerase chain reaction (PCR). “Recombinant” nucleic acids are also those that result from recombination of endogenous or exogenous nucleic acids through the natural mechanisms of cells or cells modified to allow recombination (e.g., cells modified to express Cre or other suitable recombinase), but are selected for after the introduction to the cells of nucleic acids designed to allow and make recombination probable. For example, a functionally rearranged human-antibody transgene is a recombinant nucleic acid.

The present invention also relates to antibodies (one or more) or antigen binding fragments thereof produced by the methods described above.

The antibodies or antigen binding fragment thereof, of the present invention which have binding specificity for a cyclic analog of hPTH, are useful for a variety of processes. In one aspect of the present invention, the antibodies or antigen binding fragments thereof are useful in methods such as assays or immunoassays to detect the presence of a cyclic analog of hPTH. As used herein, the term “immunoassay” is a diagnostic technique, dependent on the specificity of the antibody-antigen interaction, which is useful to detect or quantitate a substance by its action as an antigen. Typical, suitable immunoassay techniques include: enzyme immunoassays (EIA), enzyme-linked immunosorbent assays (ELISA), radioimmunoassays (RIA), and fluorescent immunoassays. Various clinical immunoassay procedures are described in Immunoassays for the 80's, A. Voller et al., (eds)., University Park, 1981.

One aspect of the present invention is a method for detecting a cyclic analog of hPTH in a sample. The method comprises combining the sample with an antibody or antigen binding fragment thereof, produced by the methods of the present invention, under conditions suitable for the formation of an immunocomplex between the antibody or antigen binding fragment thereof, and the cyclic analog of hPTH.

Another aspect of the present invention is a method for detecting a cyclic analog of HPTH in a sample, wherein said cyclic analog comprises an amino acid sequence SEQ ID NO: 1. In this aspect of the invention the sample is combined with an antibody or antigen binding fragment thereof, which has binding specificity for the cyclic analog of HPTH, under conditions suitable for the formation of an immunocomplex between the antibody and the cyclic analog of hPTH. The immunocomplex may then be detected, wherein detection of the immunocomplex indicates the presence of the cyclic analog of hPTH in the sample.

Another aspect of the present invention is a method for detecting a cyclic analog of hPTH in a sample, wherein said cyclic analog comprises an amino acid sequence selected from the group consisting: SEQ ID NO: 1, SEQ ID NO: 2, SEQ ID NO: 3, SEQ ID NO: 4, SEQ ID NO: 5, and SEQ ID NO: 10. In this aspect of the invention the sample is combined with a first antibody or antigen binding fragment thereof, which has binding specificity for an amino acid sequence comprising SEQ ID NO: 1, and a second antibody which binds the cyclic analog of hPTH under conditions suitable for the formation of an immunocomplex of the cyclic analog of hpTH with both the first antibody and the second antibody. The immunocomplex is then detected, wherein detection of the immunocomplex indicates the presence of the cyclic analog of HPTH in the sample.

Another aspect of the present invention is a method for detecting a cyclic analog of hPTH in a sample, wherein said cyclic analog comprises an amino acid sequence SEQ ID NO: 10. In this aspect of the invention the sample is combined with an antibody or antigen binding fragment thereof, which has binding specificity for an amino acid sequence comprising SEQ ID NO: 1, under conditions suitable for the formation of an immunocomplex between SEQ ID NO: 10 and the antibody. The immunocomplex may then be detected, wherein detection of the immunocomplex indicates the presence of the cyclic analog of hPTH in the sample.

Another aspect of the present invention is an immunoassay for detecting a cyclic analog of hPTH in a sample, wherein said cyclic analog comprises an amino acid sequence: SEQ ID NO: 10. In this aspect of the invention the sample is combined with a first antibody or antigen binding fragment thereof, which has binding specificity for an amino acid sequence comprising SEQ ID NO: 1, and a second antibody which binds SEQ ID NO: 10 under conditions in which SEQ ID NO: 10 binds the first antibody and the second antibody, thereby forming an immunocomplex. The immunocomplex is then detected wherein detection of the immunocomplex indicates the presence of SEQ ID NO: 10 in the sample.

For convenience an antibody or antigen binding fragment thereof which has binding specificity for a cyclic analog of hPTH is labeled as a “first antibody”. If another antibody is used in the methods of the present invention for the detection cyclic analogs of HPTH, such antibody is labeled as a “second antibody”. These labels do not confer any order on the antibodies and are used only for identification purposes.

In one aspect of the invention, either the first antibody, the second antibody or the antigen, may be immobilized on a solid phase support or carrier to facilitate isolation of the desired species. By “solid phase support” or “carrier” is intended any support capable of binding an antigen or an antibody, which may be any of various types that are known in the art such as, for example, porous materials such as nylon, glass fibers, or polymeric materials. The support material may have virtually any possible structural configuration so long as it permits the formation of an immunocomplex between the coupled molecule and its specific antibody or antigen. Thus, the support configuration may be spherical, as in a bead, or cylindrical, as in the inside surface of a test tube, or the external surface of a rod. Alternatively, the surface may be flat such as a sheet, test strip, etc. Particular supports include microtiter well plates. Those skilled in the art will know many other suitable carriers for binding antibody or antigen, or will be able to ascertain the same by use of routine experimentation.

In one embodiment, a sample comprising a cyclic analog of HPTH, is immobilized on a solid phase support. The support is then washed with suitable buffers, to remove any unbound cyclic analog of hPTH, and treated with a quantity of the first antibody. The support is then washed with the buffer a second time to remove the unbound first antibody. The immunocomplex if formed between the first antibody and the cyclic analog of hPTH is then detected, wherein detection of an immunocomplex indicates the presence of the cyclic analog of hPTH in the sample.

In another embodiment of the present invention, the first antibody, the second antibody, or the antigen may be bound to a solid phase support by conjugation with, for example, biotin or a molecule that comprises biotin. The utility of biotin, a water-soluble vitamin, arises from its ability to bind strongly to the tetrameric protein avidin, found in egg white and the tissues of birds, reptiles and amphibians, or to its chemical cousin, streptavidin. The biotin interaction with avidin is among the strongest non-covalent affinities known, exhibiting a dissociation constant of about 1.3×10-15 M (Hermanson, G. T., Bioconjugate Techniques, Academic Press, San Diego, Calif. (1996), p. 570).

In other embodiments, the conjugating molecule is biocytin and/or a biotin analog (e.g., biotin amido caproate N-hydroxysuccinimide ester, biotin-PEO4-N-hydroxysuccinimide ester, biotin 4-amidobenzoic acid, biotinamide caproyl hydrazide) and biotin derivatives (e.g., biotin-dextran, biotin-disulfide-N-hydroxysuccinimide ester, biotin-6 amido quinoline, biotin hydrazide, d-biotin-N hydroxysuccinimide ester, biotin maleimide, d-biotin p-nitrophenyl ester, biotinylated nucleotides, biotinylated amino acids such as N.epsilon.-biotinyl-1-lysine) (see, e.g., U.S. Pat. No. 5,948,624).

In a particular embodiment, avidin is immobilized on a solid phase support or carrier (e.g., an avidin-containing microtiter well plate). The subsequent interaction of the biotin with avidin can then be used to immobilize the first antibody, the second antibody, or the antigen on the solid phase support and therefore capture or isolate the desired species.

In one embodiment, the first antibody, the second antibody, or the antigen, can be labeled or unlabeled. When unlabeled, the presence of a cyclic analog of hPTH in a sample can be detected using suitable means, for example, agglutination assays. As used herein, the term “label” is a detectable moiety that possesses a specifically identifiable physical property which allows it to be distinguished from other molecules that are present in a heterologous mixture. Suitable labels include, e.g., an affinity label, an enzyme label, a fluorescent group, a chemiluminescent group, and a radioactive label.

In one embodiment the first antibody, the second antibody or the antigen is directly or indirectly labeled. In the case of indirect labeling the first antibody, the second antibody or the antigen can be used in combination with another (i.e., one or more) suitable reagent, which can be used to detect the antibody or antigen. An example of such a reagent is a labeled antibody, which recognizes and binds the first antibody, the second antibody or the antigen, and can be thus used to detect or quantitate the amount of cyclic analog of hPTH in a sample.

One of the ways in which the first antibody, the second antibody or the antigen can be directly labeled is by linking the same to an enzyme in an enzyme immunoassay (EIA), or enzyme-linked immunosorbent assay (ELISA). An enzyme, when subsequently exposed to its substrate, will react with the substrate generating a chemical moiety which can be detected, for example, by spectrophotometric, fluorometric, or by visual means (e.g., calorimetric). When a sample comprising a cyclic analog of HPTH is combined with the enzyme labeled antibodies binding occurs between the antibodies and cyclic analog of hPTH. These bound cyclic analogs of hPTH can be separated from unbound reagents and the presence of the antibody-enzyme conjugate specifically bound to the cyclic analog of hPTH can be determined, for example, by contacting the sample with a substrate of the enzyme which produces a color or other detectable change when acted on by the enzyme.

Enzymes that can be used as labels include e.g., horseradish peroxididase (HRP), alkaline phosphatase (AP), β-galactosidase (β-gal), glucose oxidase (GO), maltose binding protein and glutathione-5-transferase (see, e.g., Hermanson, G. T., Bioconjugate Techniques, Academic Press, San Diego, Calif. (1996); the entire teachings of which are incorporated herein by reference). Other suitable enzymes, proteins and/or peptides that possess one or more properties that are suitable for detection and/or imaging of the antibody can also be used as labels. In a particular embodiment the antibody is labeled with HRP.

By radioactively labeling the first antibody, the second antibody or the antigen it is possible to detect cyclic analog of hPTH through the use of a radioimmunoassay (RIA) (see, for example, Work, T. S., et al., Laboratory Techniques and Biochemistry in Molecular Biology, North Holland Publishing Company, N.Y. (1978).

The radioactive isotope can be detected by such means as the use of a gamma counter or a scintillation counter or by autoradiography. Suitable radioactive labels include, but are not limited to, iodine-131, iodine-125, bismuth-212, yttrium-90, yttrium-88, technetium-99m, copper-67, rhenium-188, rhenium-186, gallium-66, gallium-67, indium-111, indium-114m, indium-115 and boron-10 see e.g., B-phycoerythrin, R-phycoerthyrin) and derivatives of any of the foregoing (e.g., Hermanson, G. T., Bioconjugate Techniques, Academic Press, San Diego, Calif. (1996), p. 364 et seq.).

It is also possible to label the first antibody, the second antibody or the antigen with a fluorescent compound. When the fluorescent labeled antibody is exposed to light of the proper wavelength, its presence can then be detected due to fluorescence. Among the most commonly used fluorescent labeling compounds are fluorescein, fluorescein isothiocyanate, rhodamine, coumarin, phycoerythrin, phycocyanin, allophycocyanin, o-phthaldehyde and fluorescamine.

The first antibody, the second antibody or the antigen also can be detectably labeled by coupling to a chemiluminescent compound. The presence of the chemiluminescently labeled antibody is then determined by detecting the presence of luminescence that arises during the course of a chemical reaction. Examples or particularly useful chemiluminescent labeling compounds are luminol, isoluminol, theromatic acridinium ester, imidazole, acridinium salt and oxalate esters.

Likewise, a bioluminescent compound may be used to label the first antibody, the second antibody or the antigen. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In one aspect, the invention is a competition immunoassay in which a cyclic analog of hPTH, labeled with a detectable label, and an unlabeled cyclic analog of HPTH are competitively reacted with a first antibody. In an alternative competition immunoassay a cyclic analog of HPTH can be immobilized on a solid phase, incubated with a first labeled antibody, and further incubated with a first unlabeled antibody, wherein both antibodies compete for one epitope on the cyclic analog of hPTH.

In another aspect of the present invention, the antibodies or antigen binding fragments thereof of the present invention may be adapted for utilization in an immunometric assay, also known as a “sandwich” assay in which at least two antibodies are used. In a typical immunometric assay, a quantity of a second antibody (a “two-site” assay), is used, wherein the cyclic analog of HPTH can bind both the first and second antibodies. Each antibody is capable of binding an antigen epitope and avoid sterically hindering the other antibody from binding. In one embodiment both the first antibody and the second antibody can bind the cyclic region of the cyclic analog of HPTH if the second antibody does not interfere with the binding of the first antibody. In another embodiment the second antibody binds an epitope other than the cyclic region of the cyclic analog of hPTH. In yet another embodiment the second antibody binds the linear region of the cyclic analog of hPTH. In a particular embodiment the second antibody binds the N-terminal region of the cyclic analog of hPTH, for example, between about amino acids at position 1 and about amino acids at position 16 of the cyclic analog of hPTH (see for example U.S. Pat. No. 5,872,221, U.S. Pat. No. 6,030,790, and U.S. Patent Application No. 2003/0082179, the entire contents of the patents and patent application listed are incorporated herein by reference). In another embodiment the second antibody binds to the C-terminal region of the cyclic analog of HPTH, for example between about amino acids at positions 27 and about amino acids at position 31 of a cyclic analog of hPTH-(1-31), between about amino acids at positions 27 and about amino acids at position 32 of a cyclic analog of hPTH-(1-32), between about amino acids at positions 27 and about amino acids at position 33 of a cyclic analog of hPTH-(1-33), between about amino acids at positions 27 and about amino acids at position 34 of a cyclic analog of hPTH-(1-34), between about amino acids at positions 27 and about amino acids at position 84 of a cyclic analog of hPTH-(1-84).

One embodiment of the present invention is a sandwich immunoassay in which one antibody (“capture antibody”) immobilized on a solid phase support is incubated with an antigen, and further incubated with a detectably labeled antibody (“tracer” antibody) to form a “ternary” or “sandwich” structure between the capture antibody, the antigen, and the tracer antibody. The labeled antibody can then be detected by conventional means, wherein the presence of the labeled antibody on the solid phase support indicates the presence of the antigen.

The second antibody can be produced by any of the methods described above, or by the methods described in U.S. Pat. Nos. 6,689,566, 6,030,790, and 5,872,221, and U.S. Published Patent Application Nos. 2002/0110871 and 2003/0082179 the entire contents of each of which are incorporated herein by reference.

One immunometric assay embodied by the present invention is a “two-step” assay. This may be carried out as a “forward” assay or a “reverse” assay. In one embodiment the invention is a forward assay in which the second antibody bound to the solid phase support is first contacted with the sample being tested, to capture or extract the cyclic analog of hPTH from the sample by formation of a binary solid phase second antibody-cyclic analog of HPTH complex. After a suitable incubation period, the solid phase support is washed to remove the residue of the fluid sample, including unreacted cyclic analog of hPTH, if any, and then contacted with the solution containing a known quantity of labeled first antibody. After a second incubation period to permit the labeled first antibody to complex with the cyclic analog of hPTH bound to the solid support through the unlabeled second antibody, the solid phase support is washed a second time to remove the unreacted labeled first antibody, of the present invention. The labeled first antibody bound to the solid phase may then be detected, wherein the presence of labeled antibody bound to the solid phase indicates the presence of the cyclic analog of HPTH in the sample.

In another embodiment the present invention is a reverse assay, in which a solution of labeled first antibody or antigen binding fragment thereof, is combined with the sample followed by the addition of unlabeled second antibody bound to a solid phase support after a suitable incubation period. After a second incubation, the solid phase support is washed in conventional fashion to free it of the residue of the sample being tested and the solution of unreacted labeled first antibody. The labeled first antibody bound to the solid phase may then be detected, wherein the presence of labeled antibody bound to the solid phase indicates the presence of the cyclic analog of hPTH in the sample.

Other types of “sandwich” assays, which may also be useful to detect cyclic analogs of hPTH, are the so-called “simultaneous” or “one-step” assays. A particular embodiment of the present invention is a simultaneous assay. A simultaneous assay involves a single incubation step wherein the second antibody bound to the solid phase support, and the labeled first antibody, are both combined with the sample being tested at the same time. After the incubation is completed, the solid phase support is washed to remove the residue of sample and uncomplexed labeled first antibody. The labeled first antibody bound to the solid phase may then be detected, wherein the presence of labeled first antibody bound to the solid phase indicates the presence of the cyclic analog of hPTH in the sample.

In a particular embodiment the present invention is a simultaneous immunoassay in which a biotinylated second antibody bound to a streptavidin coated microtiter plate, and a HRP labeled first antibody, are both combined with the sample being tested at the same time. After the incubation is completed, the solid phase support is washed to remove the residue of sample and uncomplexed HRP labeled first antibody. The HRP labeled first antibody bound to the solid phase may then be detected, wherein the presence of the first antibody bound to the solid phase indicates the presence of the cyclic analog of hPTH in the sample.

In the methods of the present invention, whether a cyclic analog of hPTH is present in a sample may be determined qualitatively or quantitatively. A qualitative method, for example, may involve combining an enzyme labeled first antibody, a sample containing a cyclic analog of hPTH, and a second antibody and visually inspecting a color change on addition of a substrate for the enzyme bound to the first antibody, wherein a color change indicates the presence of the cyclic analog of hPTH in the sample. A quantitative method, for example, may involve combining an enzyme labeled first antibody, a sample containing a cyclic analog of hPTH, and a second antibody, and comparing the measure of the color change on addition of a substrate for the enzyme bound to the first antibody, with, for example, a standard curve obtained for a standard samples containing known quantities of cyclic analog of hPTH.

Those skilled in the art will be able to determine operative and optimal assay conditions for each determination by employing routine experimentation.

For the purposes of the present invention, the cyclic analog of hPTH which is detected by the above assays, may be present in any sample containing a cyclic analog of hPTH. For example, the sample can be a biological fluid such as, blood, serum, plasma, lymph, urine, inflammatory exudate, cerebrospinal fluid, amniotic fluid, a tissue extract or homogenate, and the like. However, the invention is not limited to assays using only these samples, it being possible for one of ordinary skill in the art to determine suitable conditions which allow the use of other samples.

In one embodiment of the invention, the sample is obtained from a subject being treated with a cyclic analog of HPTH. In a particular embodiment the sample is obtained from a subject being treated with a cyclic analog of HPTH wherein the cyclic analog comprises SEQ ID NO: 10.

One embodiment the present invention is a kit for use in detecting the presence of a cyclic analog of hPTH, comprising an antibody (one or more) or antigen binding fragment thereof, which has binding specificity for a cyclic analog of hPTH, wherein the cyclic analog comprises the amino acid sequence selected from the group comprising: SEQ ID NO: 1, SEQ ID NO: 2, SEQ ID NO: 3, SEQ ID NO: 4, SEQ ID NO: 5, or SEQ ID NO: 10. Such kits can also include one or more ancillary agents suitable for detecting the presence of a complex between the antibody of the present invention and a cyclic analog of HPTH. Washing buffers, diluents, solvents and stop solutions may also be provided in the kit.

Another embodiment of the present invention is a kit comprising an enzyme labeled first antibody (one or more) or antigen binding fragment thereof, which has binding specificity for an amino acids sequence comprising SEQ ID: NO 1, as well as a color producing substrate useful for detection of the enzyme labeled antibody. A biotinylated second antibody which binds SEQ ID NO: 10 may also be provided. Streptavidin coated microtiter plates and washing buffers, diluents, solvents and stop solutions may also be provided in the kit.

The antibodies or antigen binding fragments thereof of the present invention can be included in the kits with adjunct ingredients for example buffers (e.g., tris-hydroxymethyl aminomethane (Tris), phosphate, or carbonate), stabilizers and/or inert proteins (e.g., bovine serum albumin) enzyme substrate (e.g., HRP substrate: tetramethylbenzidine and hydrogen peroxide), acid “stop” solutions (e.g., sulfuric acid), and controls and/or standards containing a known concentration of the antigen being tested. The antibodies can be provided in combination with the adjunct ingredients, or the adjunct ingredients can be separately provided, for combination by the user.

The antibody or antigen binding fragment thereof of the present invention can be provided in combination with second antibodies specific for other epitopes of the cyclic analog of hPTH. Where a second antibody capable of binding to a second epitope on the cyclic analog of hPTH is employed, such antibody can be provided in the kit, for instance in combination with the first antibody or in a separate vial or container. In a particular embodiment the second antibody is conjugated to biotin.

A support matrix suitable for a method or assay to detect the presence of a cyclic analog of hPTH can also be provided in the kit. In a particular embodiment the support matrix comprises a microplate with twelve by eight strips (ninety six microwells in total). In a particular embodiment the support matrix comprises a streptavidin coated microplate.

The antibodies and/or ancillary reagents of the kit can be packaged separately or together within suitable containment means (e.g., bottle, box, envelope, tube). When the kit comprises a plurality of individually packaged components, the individual packages can be contained within a single larger containment means (e.g., bottle, box, envelope, tube).

In one embodiment, the kit of the present invention may be adapted to be employed in an automated assay system to determine the concentration of cyclic analog of hPTH.

In another embodiment the present invention is an antigenic peptide useful in inducing an animal to produce an antibody which has binding specificity for a cyclic analog of hPTH. Such antigenic peptides may be prepared by any of a variety of methods well-known in the art including synthesis by conventional methods, such as solid-phase chemical synthesis or by recombinant technology. In a particular embodiment the technique of solid phase synthesis developed by R. B. Merrifield (Solid-Phase Peptide Synthesis, Advances in Enzymology 32, 221-296 1969), the entire contents of which are incorporated herein by reference, is used for the synthesis of the antigenic peptides. The strategy is based on having the carboxyl-terminus amino acid of the peptide attached to a solid support. Successive amino acids are then added in high yield. The N-terminal a-amino group is protected in such a way that this protecting group can be removed without removal of the peptide from the solid support. The chemistry used here involves a modification of the original Merrifield method, referred to as the Fmoc approach. The Fmoc (fluorenylmethoxycarbonyl) group can be removed by mild alkaline conditions, which leaves the alkali stable side-chain protecting groups and the link to the support untouched. This technique is described by E. Atherton and R. C. Sheppard, Solid Phase Peptide Synthesis; a Practical Approach, IRL Press new York, N.Y., the entire contents of which are incorporated herein by reference.

In one embodiment the antigenic peptide consists of the amino acid sequence SEQ ID NO: 1. In another embodiment, the antigenic peptide consists of the amino acid sequence SEQ ID NO: 2. In a particular embodiment the antigenic peptide consists of the amino acid sequence SEQ ID NO: 3. In a particular embodiment the antigenic peptide consists of the amino acid sequence SEQ ID NO: 12. In another embodiment particular peptides which can be used to generate antibodies, which have binding specificity for cyclic analogs of hPTH, comprise at least four consecutive amino acids comprising at least one amino acid from the cyclic region of the cyclic analog of hPTH. In another embodiment, peptides which can be used to generate antibodies, which have binding specificity for cyclic analogs of hPTH, comprise at least four consecutive amino acids comprising at least one amino acid from: positions 1 through 5 of SEQ ID NO: 1, positions 1 through 5 of SEQ ID NO: 2, positions 5 through 9 of SEQ ID NO: 3, positions 22 through 26 of SEQ ID NO: 4, positions 22 through 26 of SEQ ID NO: 5, or positions 22 through 26 of SEQ ID NO: 10.

The antigenic peptides of the present invention may be optionally coupled to a carrier molecule to increase the immunogenic properties of the antigenic peptides. In a particular embodiment the carrier molecule is selected from the group: keyhole limpet hemocyanin (KLH), bovine serum albumin (BSA), hemocyanin, thyroglobulin, mouse serum albumin, or ovalbumin. In a particular embodiment the carrier molecule is mariculture keyhole limpet hemocyanin (mcKLH).

Coupling of antigenic peptides to carrier proteins may be achieved by the use of heterobifunctional cross-linkers, homobifunctional cross-linkers, the Mannich reaction and many other methods. In one embodiment M-Maleimidobenzoyl-N-hydroxysuccinimide ester (MBS) is used to link the antigenic peptides to carrier proteins. The peptides may optionally be modified at the N-terminal or C-terminal to facilitate the binding to the carrier molecule. Such modification includes the addition of a cysteine residue to the N- or C-terminal.

In an alternative embodiment, recombinant peptides may be generated as fusion proteins, to increase the immunogenic properties of the antigenic peptides.

The present invention is further illustrated by the following examples, which are not intended to be limiting in any way.

### EXAMPLE 1

### Synthesis and Purification of cyclic hPTH-(17-31)-amide analogs [Cys¹⁷, Leu²⁷] cyclo(Gu²²-Lys²⁶)hPTH(17-31)NH₂

| | |
|---|---|
| 20 30 | |
| Cys-Met-Glu-Arg-Val-Glu-Trp-Leu-Arg-Lys-Leu-Leu-Gln-Asp-Val-NH₂ | (SEQ ID NO:12) |

The amino acid α-amino groups were protected by 9-fluorenyl-methoxycarbonyl (Fmoc) during coupling. Couplings were performed with a mixture of hydroxybenzotriazole (HOBt), 2-(1H-benzotriazole-1-yl) 1,1,3,3-tetramethyluronium tetrafluoroborate (TBTU), and collidine in 1:1 dimethylformamide (DMF)/dichloromethane (DCM). A 4-fold excess of activated amino acids was used with double coupling on addition of: Cys-1, Glu-3, Arg-4, Val-5, Leu-8, Leu-11, Leu-12, Gln-13, Asp-14, Val-15. The coupling time for Arg additions was increased from 30 to 60 minutes. The solid support was Tentagel R RAM (Peptides International) (substitution, 0.21 mmol/g. The synthesis was performed on a PerSeptive Biosystems Model 9050 Plus automated peptide synthesizer. Side chain protections were as follows: N^{G}-2,2,4,6,7-pentamethyldihydrobenzofuran-5-sulfonyl-L-arginine; Glu-4, Asp-14 (t-butyl); Cys-1, Gln-13 (trityl); Lys-10(Boc); Trp-7 (t-butyloxycarbonyl), Glu-6(OAll), Lys-10(All). Upon completion of the synthesis, the peptide resin was removed from the column to a reaction vial (Minivial, Applied Science) the All and Alloc groups were removed by suspension in 1.7 ml of a solution of tetrekis(triphenylphosphine)palladium(0) (0.24 mmol), 5% acetic acid, and 2.5% NMM in DCM under argon, and then shaken at 20° C. for 6 h. The peptide resin was then washed with 0.5% diethyldithiocarbamate and 0.5% N-methylmorpholine (NMM) in DMF (50 ml), followed by DMF (50 ml) and DCM (50 ml). The peptide (0.06 mmol) was cyclized by shaking with 0.06 mmol of 7-azabenzotriazol-1-yloxy)tris(pyrrolidino)-phosphonium hexafluorophosphate(PyAOP)/HOBt/0.12 mmol NMM in 2 ml of DMF for 14 h at 20° C.

After Fmoc removal from the N-terminus, the peptide resin was washed with DCM, and then cleaved from the resin by shaking with 7.5 ml of reagent K (6.19 ml TFA, 0.38 ml each of water, 90% phenol/water, and thioanisole, and 0.19 ml of 1,2-ethanedithiol) for 4 hr at 20° C. The cleaved peptide mixture was removed by filtration, and precipitated by addition to t-butylmethylether. The precipitate was collected by centrifugation, washed 2× with t-butylmethylether, and then dried by vacuum centrifugation. The crude product was dissolved in 14 ml of 15% acetonitrile/water, 0.1% TFA and chromatographed on a Vydac CI8-column (10μ, 1×25 cm). The product was eluted with a 1%/min. gradient of acetonitrile (15-40%) in 0.1% TFA in water. The purity of the final product was estimated by analytical HPLC on a Vydac C18 column (10μ, 0.4˜2.5 cm), and by MALDI-TOF MS. For [Cys¹⁷, Leu²⁷] cyclo(Glu²²-Lys²⁶) hPTH-(17-31)-NH₂: MW=1900.0 (M+)

### EXAMPLE 2

### Synthesis and Purification of cyclic hPTH-(17-31)-amide analogs [Leu²⁷, Cys³²]cyclo(Glu²² Lys²⁶)hPTH(17-32)NH₂

| | |
|---|---|
| 20 30 | |
| Ser-Met-Glu-Arg-Val-Glu-Trp-Leu-Arg-Lys-Leu-Leu-Gln-Asp-Val-Cys-NH₂ | (SEQ ID NO:13) |

The peptide was synthesized by an equivalent protocol to Example 1. The molecular weight was MW=1986.8 (M+).

### EXAMPLE 3

### Preparation of a keyhole limpet hemocyanin (KLH) conjugate with [Cys¹⁷, Leu²⁷] cyclo(Glu²²-Lys²⁶) hPTH-(17-31)—NH₂

The [Cys¹⁷, Leu²⁷] cyclo(Glu²²-Lys²⁶) hPTH-(17-31)—NH₂ (SEQ ID NO: 12) (2 mg) was conjugated to maleimide-activated KLH (2 mg) (Pearce Chemicals) in the presence of 80 mM sodium phosphate, 0.1 M EDTA, 0.9 M NaCl, pH 7.2 for 2 hr at room temperature. Small reactants were removed by passage through a desalting column equilibrated with 80 mM sodium phosphate, 0.9 M NaCl, pH 7.2.

### EXAMPLE 4

### Preparation and purification of antibodies to [Cys¹⁷, Leu²⁷] cyclo(Glu²²-Lys²⁶) hPTH-(17-31)—NH₂

Six New Zealand white female rabbits were injected initially with 100 μg each of the peptide-KLH conjugate suspended in phosphate buffered saline, pH 7.2, in a 1:1 emulsion with Freund's complete adjuvant, then subsequently boosted at 4 and 8 weeks with the same conjugate amount but emulsified with Freund's incomplete adjuvant. Blood samples were collected after eight weeks and tested for their sensitivity and specificity for [Leu²⁷]cyclo[Glu²²-Lys²⁶]hPTH(1-31)NH₂ (SEQ ID NO: 10).

The antisera obtained from the immunized animals was then affinity purified using a Protein A gel packed column (Bio-Rad Laboratories, Hercules, Calif. 94547, USA). The antiserum was loaded on to the column slowly to allow the antibodies to bind to the Protein-A gel. Unbound proteins and materials were washed away using a 0.01 M phosphate buffered saline. The antibodies were then eluted with an elution buffer of 0.1 M glycine-HCl (pH 2.5). The antibody fractions were collected, pooled and dialyzed against 0.01 M phosphate buffered saline.

The antibodies were then conjugated with horse radish peroxidase (HRP) with a very high specific enzyme activity. The coupling reaction was carried out according to the two-step glutaraldehyde method (Avermeas S, Temynck T., “Peroxidase labeled antibody and Fab conjugates with enhanced intracellular penetration”, Immunochemistry, 8:1175-9, (1971)), developed at Epitope Diagnostics, Inc. (San Diego, Calif. 92126, USA). The conjugated antibody was diluted with a bovine serum albumin based matrix and stored at 2-8° C. or −20° C.

### EXAMPLE 5

### Preparation and purification of antibodies to the non-cyclic portion of [Cys¹⁷, Leu²⁷] cyclo(Glu²²-Lys²⁶) hPTH-(1-31)—NH₂

Goats were injected with 100 μg each of hPTH-(1-34) peptide conjugated to bovine thyroglobulin, which is suspended in phosphate buffered saline, pH 7.2, in a 1:1 emulsion with Freund's complete adjuvant, then subsequently boosted every 4 weeks for an extended 12 months. Blood samples were collected after 3 months and tested for their binding capabilities.

The antisera obtained from the immunized animals was affinity purified using an antigen specific gel packed column. The [Cys 7, Leu²⁷] cyclo(Glu²²-Lys²⁶) hPTH-(1-31)—NH₂ was conjugated to a CNBr-column (Bio-Rad Laboratories, Hercules, Calif. 94547, USA) according to manufacturer's instruction. The antiserum to hPTH-(1-34) was loaded on to the column slowly to allow the antibodies to bind to the linear portion of [Cys¹⁷, Leu²⁷] cyclo(Glu²²-Lys²⁶) hPTH-(1-31)—NH₂ conjugated gel. Unbound proteins and non-specific antibodies were washed away using a 0.01 M phosphate buffered saline. The anti-[Cys¹⁷, Leu²⁷] cyclo(Glu²²-Lys²⁶) hPTH-(1-31)—NH₂ specific antibodies were then eluted with an elution buffer of 0.1M glycine-HCl (pH 2.5). The antibody fractions were collected, pooled and dialyzed against 0.01M phosphate buffered saline.

### EXAMPLE 6

### Biotinylation of anti-non cyclic portion of [Cys¹⁷, Leu²⁷] cyclo(Glu²²-Lys²⁶) hPTH-(1-31)—NH₂ antibody

The antibodies from Example 5 were biotinylated by mixing one portion of antibody to 20 portions of activated NHS-Biotin (mol:mol) (Sigma, St Luis, Mo. 63178, USA). After incubation at room temperature for 18-20 hours, the antibody was dialyzed intensively against 0.01M phosphate buffered saline. The final biotinylated antibody was diluted in a phosphate buffered saline with bovine serum albumin to a desired concentration. This antibody was stored at 2-8° C.

### EXAMPLE 7

### Sandwich ELISA for [Leu²⁷]cyclo(Glu²²-Lys²⁶)hPTH-(1-31) (SEQ ID NO: 10)

Streptavidin was weighed and diluted to 20 mg/L with phosphate buffer, 0.2 ml of this solution was added to each well of Corning® 96 Well Polystyrene Microplate. The plates were incubated at room temperature for 18-22 hours. The plates were then washed and a stabile/blocking buffer containing BSA was added. The plates were again incubated at room temperature for four hours. The plates were finally dried at <30% humidity.

[Leu²⁷]cyclo(Glu²²-Lys²⁶)hPTH-(1-31) (SEQ ID NO: 10) peptide standards were prepared as follows. The peptide was diluted with a bovine serum albumin and normal bovine serum based buffer matrix to a final concentration of 1600 pg/ml, 400 pg/ml, 100 pg/ml, 25 pg/ml and 6 pg/ml. For the purpose of the ELISA a buffer matrix was used as the zero standard. 100 μL of each of the peptide standards was added into designated wells of a Corning® 96 Well Polystyrene Microplate coated with streptavidin as described above. 100 μL of an antibody mixture which contained 120 ng of affinity purified anti-N-terminal [Leu²⁷]cyclo(Glu²²-Lys²⁶)hPTH-(1-31) (SEQ ID NO: 10) antibody conjugated with biotin-NHS and 20 ng affinity purified anti-C-terminal [Leu²⁷]cyclo(Glu²²-Lys²⁶)hPTH-(1-31) (SEQ ID NO: 10) antibody labeled with horseradish peroxidase (HRP), was then added into each well.

The above antigen and antibodies were incubated in the streptavidin coated well for 3 hours at room temperature with shaking at 170 rpm. After incubation, each well was washed with an ELISA wash buffer. 200 μL of tetra methyl benzidine (TMB) was then added into each well. The wells were incubated for 20 min at room temperature and then 100 μL of a stop solution was added into each well. The Microplates were read with a microtiter plate reader (VERSAmax™, Molecular Device, Inc.) at an absorption wavelength of 450 nm.

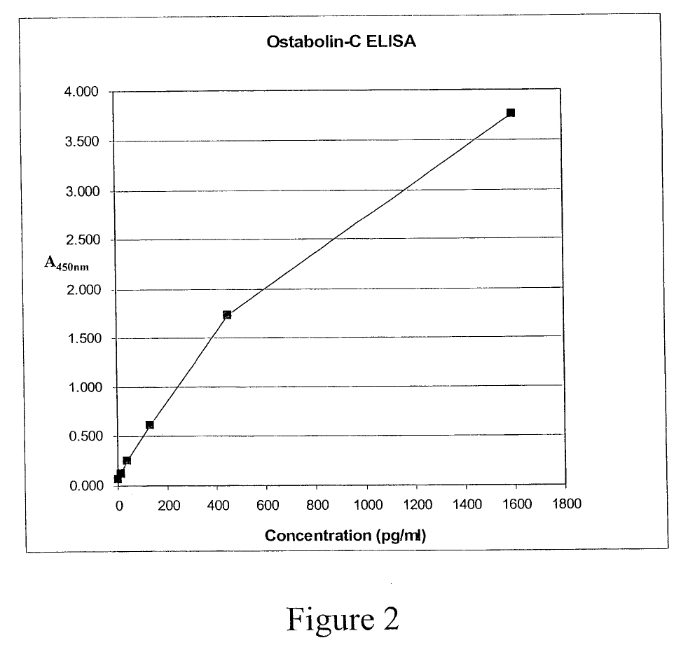

A standard curve was obtained by plotting the optical density (OD) at 450 nm against the correspondent Ostabolin-C standard concentration. A dose responsive standard curve was obtained using above two-site “sandwich” ELISA method (FIG. 2).

### EXAMPLE 8

### Binding Selectivity of the Antibodies for Cyclic Analogs of hPTH over the Linear HPTH Analogs

[Leu²⁷]cyclo(Glu²²-Lys²⁶)hPTH-(1-31) (SEQ ID NO: 10) and the following linear hPTH-analogs: hPTH-(1-84), hPTH-(1-31), and hPTH-(1-34) (purchased from Bachem, Inc.), were diluted individually with a bovine serum albumin based buffer matrix to a final concentration of 10,000 pg/ml, 1,000 pg/ml and 100 pg/ml in separated containers. These artificial peptide-containing samples were then measured in an [Leu²⁷]cyclo(Glu²²-Lys²⁶)hPTH-(1-31) (SEQ ID NO: 10) two-site “sandwich” ELISA as described in Example 5.

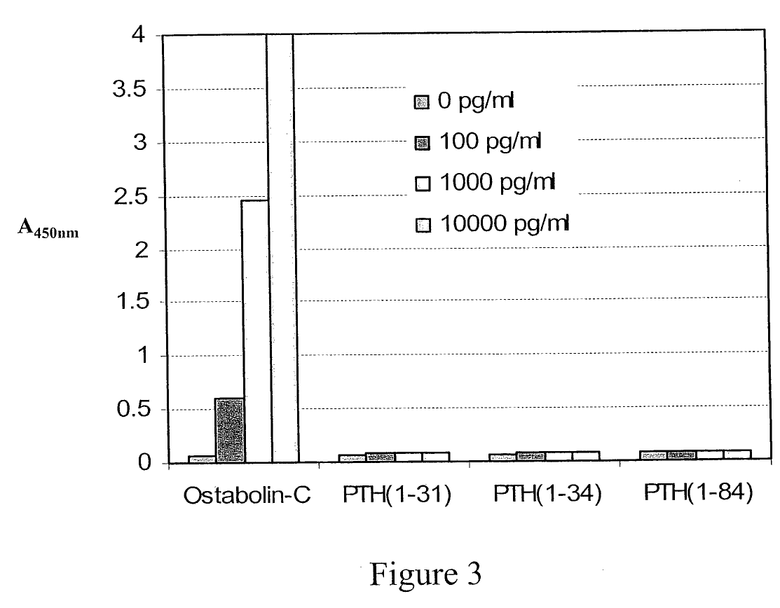

The OD at 450 nm values were read by a microtiter plate reader (VERSAmaX™, Molecular Device, Inc.) (FIG. 3).

The assay detected [Leu²⁷]cyclo(Glu²²-Lys²⁶)hPTH-(1-31) (SEQ ID NO: 10) peptides in a dose responsive manner. However, the [Leu²⁷]cyclo(Glu²²-Lys²⁶)hPTH-(1-31) (SEQ ID NO: 10) assay was not able to detect any other hPTH analogs including hPTH-(1-84), hPTH-(1-34) and hPTH-(1-31) peptides up to a concentration of 10,000 pg/ml, wherein, all the results at OD 450 mm were similar or close to that of the buffer matrix. Therefore, the antibodies and assays are specific for measuring [Leu²⁷]cyclo(Glu²²-Lys²⁶)hPTH-(1-31) (SEQ ID NO: 10) without any cross-reaction with linear hPTH-(1-84), hPTH-(1-34) and hPTH-(1-31).

While this invention has been particularly shown and described with references to particular embodiments thereof, it will be understood by those skilled in the art that various changes in form and details may be made therein without departing from the scope of the invention encompassed by the appended claims.

1. An antibody or antigen binding fragment thereof which has binding specificity for a cyclic analog of human parathyroid hormone (hPTH).

2. The antibody or antigen binding fragment thereof of claim 1, which has binding specificity for a cyclic analog of human parathyroid hormone (hPTH), wherein the cyclic analog comprises an amino acid sequence: Glu-Trp-Leu-Arg-Lys (SEQ ID NO: 1) which is cyclized between Glu¹ and Lys⁵.

3. The antibody or antigen binding fragment thereof of claim 2, wherein the cyclic analog comprises an amino acid sequence: Glu-Trp-Leu-Arg-Lys-Leu-Leu (SEQ ID NO: 2) which is cyclized between Glu¹ and Lys⁵.

4. The antibody or antigen binding fragment thereof of claim 3, wherein the cyclic analog comprises an amino acid sequence: Met-Glu-Arg-Val-Glu-Trp-Leu-Arg-Lys-Leu-Leu-Gln-Asp-Val (SEQ ID NO: 3) which is cyclized between Glu⁵ and Lys⁹.

5. The antibody or antigen binding fragment thereof of claim 4, wherein the cyclic analog comprises an amino acid sequence: R—NH-Xaa1-Val-Ser-Glu-Ile-Gln-Leu-Xaa8-His-Asn-Leu-Gly-Xaa13-Xaa14-Xaa15-Xaa16-Xaa17-Met-Glu-Arg-Val-Glu-Trp-Leu-Arg-Lys-Leu-Leu-Gln-Asp-Val-Y (SEQ ID NO: 4), wherein:
the cyclic analog is cyclized between Glu²² and Lys²⁶;
R is a hydrogen or any linear or branched chain alkyl, acyl or aryl group;
Xaa1 is serine, alanine, norleucine, or α-aminoisobutyric acid;
Xaa8 is methionine, norisoleucine, norleucine, or a hydrophobic amino acid;
Xaa13 is lysine, ornithine, glutamic acid, aspartic acid, cysteine, or homocysteine;
Xaa14 is histidine or a water soluble amino acid;
Xaa15 is leucine or a water soluble amino acid;
Xaa16 is asparagine or a water soluble amino acid;
Xaa17 is serine or a water soluble amino acid; and
Y is X (SEQ ID NO: 4), His-X (SEQ ID NO: 14), His-Asn-X (SEQ ID NO: 15), or His-Asn-Phe-X (SEQ ID NO: 16); where X is NH₂ or OH.

6. The antibody or antigen binding fragment thereof of claim 4, wherein the cyclic analog comprises an amino acid sequence: H—NH-Ser-Val-Ser-Glu-Ile-Gln-Leu-Met-His-Asn-Leu-Gly-Lys-Xaa14-Xaa15-Xaa16-Xaa17-Met-Glu-Arg-Val-Glu-Trp-Leu-Arg-Lys-Leu-Leu-Gln-Asp-Val-Y (SEQ ID NO: 5), wherein:
the cyclic analog is cyclized between Glu²² and Lys²⁶;
Xaa14 is histidine or lysine;
Xaa15 is leucine, lysine, or arginine;
Xaa16 is asparagine, ornithine, homocitrulline, aspartic acid, arginine, lysine, d-lysine, serine, or glycine;
Xaa17 is serine, glutamic acid, lysine, aspartic acid, ornithine, cysteine, homocysteine, or arginine; and
Y is X (SEQ ID NO: 5), His-X (SEQ ID NO: 17), His-Asn-X (SEQ ID NO: 18), or His-Asn-Phe-X (SEQ ID NO: 19); where X is NH₂ or OH.

7. The antibody or antigen binding fragment thereof of claim 6, wherein:

| | |
|---|---|
| a) | Xaa14-Xaa17 is: His-Lys-Lys-Lys and Y is: |
| | |
|---|---|
| i) | X (SEQ ID NO:6); |
| ii) | His-X (SEQ ID NO:20); |
| iii) | His-Asn-X (SEQ ID NO:21); or |
| iv) | His-Asn-Phe-X (SEQ ID NO:22); |
| | |
|---|---|
| b) | Xaa14-Xaa17 is His-Leu-Lys-Lys and Y is: |
| | |
|---|---|
| i) | X (SEQ ID NO:7); |
| ii) | His-X (SEQ ID NO:23); |
| iii) | His-Asn-X (SEQ ID NO:24); or |
| iv) | His-Asn-Phe-X (SEQ ID NO:25); |
| | |
|---|---|
| c) | Xaa14-Xaa17 is Lys-Lys-Lys-Lys and Y is: |
| | |
|---|---|
| i) | X (SEQ ID NO:8); |
| ii) | His-X (SEQ ID NO:26); |
| iii) | His-Asn-X (SEQ ID NO:27); or |
| iv) | His-Asn-Phe-X (SEQ ID NO:28); or |
| | |
|---|---|
| d) | Xaa14-Xaa17 is His-Leu-Lys-Ser and Y is: |
| | |
|---|---|
| i) | X (SEQ ID NO:9); |
| ii) | His-X (SEQ ID NO:29); |
| iii) | His-Asn-X (SEQ ID NO:30); or |
| iv) | His-Asn-Phe-X (SEQ ID NO:31). |

8. The antibody or antigen binding fragment thereof of claim 4, wherein the cyclic analog comprises an amino acid sequence: [Leu²⁷]cyclo(Glu²²-Lys²⁶)hPTH-(1-31)NH₂ (SEQ ID NO: 10).

9. A method of producing an antibody or antigen binding fragment thereof, which has binding specificity for a cyclic analog of human parathyroid hormone (hPTH), wherein the cyclic analog comprises an amino acid sequence: Glu-Trp-Leu-Arg-Lys (SEQ ID NO: 1), which is cyclized between Glu¹ and Lys⁵, comprising the steps of:
(a) administering an antigenic peptide comprising SEQ ID NO: 1 to an animal, under conditions in which an antibody which has binding specificity for the cyclic analog of hPTH is produced in the animal; and
(b) isolating the antibody or antigen binding fragment thereof from the animal.

10. The method of claim 9, wherein the antigenic peptide comprises an amino acid sequence selected from the group consisting of: Glu-Trp-Leu-Arg-Lys-Leu-Leu (SEQ ID NO: 2) which is cyclized between Glu¹ and Lys⁵ and Met-Glu-Arg-Val-Glu-Trp-Leu-Arg-Lys-Leu-Leu-Gln-Asp-Val (SEQ ID NO: 3) which is cyclized between Glu⁵ and Lys⁹.

11. The method of claim 9, wherein the antigenic peptide comprises an amino acid sequence selected from the group consisting of: SEQ ID NO: 4, SEQ ID NO: 5, SEQ ID NO: 10, SEQ ID NO: 12, or SEQ ID NO: 13.

12. The method of claim 9, wherein the antigenic peptide is coupled to a carrier.

13. The method of claim 12, wherein the carrier is mariculture keyhole limpet hemocyanin (mcKLH).

14. The method of claim 9, wherein the antibody is a monoclonal antibody.

15. The method of claim 9, wherein the antibody is a polyclonal antibody.

16. An antibody or antigen binding fragment thereof produced by the method of claim 9.

17. The method of claim 9, wherein the cyclic analog comprises an amino acid sequence: [Leu²⁷]cyclo(Glu²²-Lys²⁶)hPTH-(1-31)NH₂ (SEQ ID NO: 10), comprising the steps of:
(a) administering an antigenic peptide comprising the amino acid sequence: Cys-Met-Glu-Arg-Val-Glu-Trp-Leu-Arg-Lys-Leu-Gin-Val-NH₂ (SEQ ID NO: 12), which is cyclized between Glu⁶ and Lys¹⁰ to an animal, under conditions in which an antibody to SEQ ID NO: 12 is produced in the animal; and
(b) isolating the antibody or antigen binding fragment thereof from the animal.

18. An antibody or antigen binding fragment thereof produced by the method of claim 17.

19. A method for detecting a cyclic analog of human parathyroid hormone (hPTH) in a sample, wherein said cyclic analog comprises an amino acid sequence: Glu-Trp-Leu-Arg-Lys (SEQ ID NO: 1), which is cyclized between Glu¹ and Lys⁵, comprising the steps of:
(a) combining the sample with an antibody or antigen binding fragment thereof, which has binding specificity for the cyclic analog of hPTH, under conditions suitable for formation of an immunocomplex between the antibody and the cyclic analog of HPTH; and
(b) detecting the immunocomplex;
wherein, detection of the immunocomplex indicates the presence of the cyclic analog of hPTH in the sample.

20. The method of claim 19, wherein the sample is further combined with a second antibody which binds the cyclic analog of HPTH, under conditions in which the first antibody and the second antibody bind the cyclic analog of hPTH, thereby forming an immunocomplex.

21. The method of claim 20, wherein the second antibody binds a non-cyclic region of the cyclic analog of HPTH.

22. The method of claim 21, wherein the non-cyclic region is an N-terminal region of the cyclic analog of HPTH.

23. The method of claim 20, wherein the cyclic analog of HPTH comprises an amino acid sequence selected from the group consisting of: Glu-Trp-Leu-Arg-Lys-Leu-Leu (SEQ ID NO: 2) which is cyclized between Glu¹ and Lys⁵ and Met-Glu-Arg-Val-Glu-Trp-Leu-Arg-Lys-Leu-Leu-Gln-Asp-Val (SEQ ID NO: 3) which is cyclized between Glu⁵ and Lys⁹.

24. The method of claim 20, wherein the cyclic analog of hPTH comprises an amino acid sequence: R—NH-Xaa1-Val-Ser-Glu-Ile-Gln-Leu-Xaa8-His-Asn-Leu-Gly-Xaa13-Xaa14-Xaa15-Xaa16-Xaa17-Met-Glu-Arg-Val-Glu-Trp-Leu-Arg-Lys-Leu-Leu-Gln-Asp-Val-Y (SEQ ID NO: 4), wherein:
the cyclic analog is cyclized between Glu²² and Lys²⁶;
R is a hydrogen or any linear or branched chain alkyl, acyl or aryl group;
Xaa1 is serine, alanine, norleucine, or α-aminoisobutyric acid;
Xaa8 is methionine, norisoleucine, norleucine, or a hydrophobic amino acid;
Xaa13 is lysine, ornithine, glutamic acid, aspartic acid, cysteine, or homocysteine;
Xaa14 is histidine or a water soluble amino acid;
Xaa15 is leucine or a water soluble amino acid;
Xaa16 is asparagine or a water soluble amino acid;
Xaa17 is serine or a water soluble amino acid; and
Y is X (SEQ ID NO: 4), His-X (SEQ ID NO: 14), His-Asn-X (SEQ ID NO: 15), or His-Asn-Phe-X (SEQ ID NO: 16); where X is NH₂ or OH.

25. The method of claim 20, wherein the cyclic analog of hPTH comprises an amino acid sequence: H—NH-Ser-Val-Ser-Glu-Ile-Gln-Leu-Met-His-Asn-Leu-Gly-Lys-Xaa14-Xaa15-Xaa16-Xaa17-Met-Glu-Arg-Val-Glu-Trp-Leu-Arg-Lys-Leu-Leu-Gln-Asp-Val-Y (SEQ ID NO: 5), wherein:
the cyclic analog is cyclized between Glu²² and Lys²⁶;
Xaa14 is histidine or lysine;
Xaa15 is leucine, lysine, or arginine;
Xaa16 is asparagine, ornithine, homocitrulline, aspartic acid, arginine, lysine, d-lysine, serine, or glycine;
Xaa17 is serine, glutamic acid, lysine, aspartic acid, ornithine, cysteine, homocysteine, or arginine; and
Y is X (SEQ ID NO: 5), His-X (SEQ ID NO: 17), His-Asn-X (SEQ ID NO: 18), or His-Asn-Phe-X (SEQ ID NO: 19); where X is NH₂ or OH.

26. The method of claim 25, wherein:

| | |
|---|---|
| a) | Xaa14-Xaa17 is: His-Lys-Lys-Lys and Y is: |
| | |
|---|---|
| i) | X (SEQ ID NO:6); |
| ii) | His-X (SEQ ID NO:20); |
| iii) | His-Asn-X (SEQ ID NO:21); or |
| iv) | His-Asn-Phe-X (SEQ ID NO:22); |
| | |
|---|---|
| b) | Xaa14-Xaa17 is His-Leu-Lys-Lys and Y is: |
| | |
|---|---|
| i) | X (SEQ ID NO:7); |
| ii) | His-X (SEQ ID NO:23); |
| iii) | His-Asn-X (SEQ ID NO:24); or |
| iv) | His-Asn-Phe-X (SEQ ID NO:25); |
| | |
|---|---|
| c) | Xaa14-Xaa17 is Lys-Lys-Lys-Lys and Y is: |
| | |
|---|---|
| i) | X (SEQ ID NO:8); |
| ii) | His-X (SEQ ID NO:26); |
| iii) | His-Asn-X (SEQ ID NO:27); or |
| iv) | His-Asn-Phe-X (SEQ ID NO:28); or |
| | |
|---|---|
| d) | Xaa14-Xaa17 is His-Leu-Lys-Ser and Y is: |
| | |
|---|---|
| i) | X (SEQ ID NO:9); |
| ii) | His-X (SEQ ID NO:29); |
| iii) | His-Asn-X (SEQ ID NO:30); or |
| iv) | His-Asn-Phe-X (SEQ ID NO:31). |

27. The method of claim 20, wherein the cyclic analog of hPTH comprises an amino acid sequence: [Leu²⁷]cyclo(Glu²²-Lys²⁶)hPTH-(1-31)NH₂ (SEQ ID NO: 10).

28. The method of claim 20, wherein the first antibody is labeled with a horseradish peroxidase (HRP) enzymatic marker.

29. The method of claim 20, wherein the second antibody is bound to biotin.

30. The method of claim 20, wherein the sample is combined with the first and second antibodies simultaneously.

31. The method of claim 20, wherein the sample is combined with the first and second antibodies sequentially.

32. The method of claim 20, wherein the sample is obtained from a subject being treated with the cyclic analog of hPTH.

33. The method of claim 19, wherein the cyclic analog comprises an amino acid sequence: [Leu²⁷]cyclo(Glu²²-Lys²⁶)hPTH-(1-31)NH₂ (SEQ ID NO: 10).

34. The method of claim 33, wherein the sample is further combined with a second antibody which binds: [Leu²⁷]cyclo(Glu²²-Lys²⁶)hPTH-(1-31)NH₂ (SEQ ID NO: 10), under conditions in which the first antibody and the second antibody bind to SEQ ID NO: 10, thereby forming an immunocomplex.

35. The method of claim 34, wherein the first antibody is labeled with a horseradish peroxidase (HRP) enzymatic marker.

36. The method of claim 35, wherein the second antibody is bound to biotin.

37. The method of claim 36, wherein the sample is combined with the first and second antibodies simultaneously.

38. The method of claim 36, wherein the sample is combined with the first and second antibodies sequentially.

39. A method for detecting a cyclic analog of human parathyroid hormone (hPTH) in a sample, wherein said cyclic analog comprises an amino acid sequence: Glu-Trp-Leu-Arg-Lys (SEQ ID NO: 1), which is cyclized between Glu¹ and Lys⁵, comprising the steps of:
(a) combining the sample, a first antibody or antigen binding fragment thereof which has binding specificity for the cyclic analog of hPTH, and a second antibody or antigen binding fragment thereof which has binding specificity for the cyclic analog of hPTH, under conditions in which the first antibody and the second antibody compete for an epitope on the cyclic analog of hPTH;
(b) forming an immunocomplex of the cyclic analog of hPTH with either the first or second antibody; and
(c) detecting the immunocomplex;
wherein, detection of the immunocomplex indicates the presence of the cyclic analog of hPTH in the sample.

40. A kit comprising an antibody or antigen binding fragment thereof which has binding specificity for a cyclic analog of human parathyroid hormone (hPTH), wherein the cyclic analog comprises an amino acid sequence: Glu-Trp-Leu-Arg-Lys (SEQ ID NO: 1) which is cyclized between Glu¹ and Lys⁵.

41. The kit of claim 40, wherein the cyclic analog of HPTH comprises an amino acid sequence selected from the group consisting of: Glu-Trp-Leu-Arg-Lys-Leu-Leu (SEQ ID NO: 2) which is cyclized between Glu¹ and Lys⁵ and Met-Glu-Arg-Val-Glu-Trp-Leu-Arg-Lys-Leu-Leu-Gln-Asp-Val (SEQ ID NO: 3) which is cyclized between Glu⁵ and Lys⁹.

42. The kit of claim 40, wherein the cyclic analog of HPTH comprises an amino acid sequence selected from the group consisting of: SEQ ID NO: 4, SEQ ID NO: 5, or SEQ ID NO: 10.

43. The kit of claim 40, wherein the antibody is coupled with a detectable label.

44. The kit of claim 43, further comprising a reagent for detecting said label.

45. The kit of claim 44, further, comprising:
(a) a second antibody, which binds a cyclic analog of hPTH; and
(b) washing buffers, diluents, solvents and stop solutions.

46. The kit of claim 40, comprising:
(a) an enzyme labeled first antibody or antigen binding fragment thereof which has binding specificity for a cyclic analog of Hpth;
(b) a biotinylated second antibody, which binds: [Leu²⁷]cyclo(Glu²²-Lys²⁶)hPTH-(1-31)NH₂ (SEQ ID NO: 10);
(c) a color-producing substrate solution for use as a substrate for the enzyme of step (a);
(d) streptavidin coated microtiter plates; and
(e) washing buffers, diluents, solvents and stop solutions.

47. An antigenic peptide consisting of an amino acid sequence: Glu-Trp-Leu-Arg-Lys (SEQ ID NO: 1) which is cyclized between Glu¹ and Lys⁵.

48. An antigenic peptide consisting of an amino acid sequence: Glu-Trp-Leu-Arg-Lys-Leu-Leu (SEQ ID NO: 2) which is cyclized between Glu¹ and Lys⁵.

49. An antigenic peptide consisting of an amino acid sequence: Met-Glu-Arg-Val-Glu-Trp-Leu-Arg-Lys-Leu-Leu-Gln-Asp-Val (SEQ ID NO: 3) which is cyclized between Glu⁵ and Lys⁹.

50. An antigenic peptide consisting of an amino acid sequence: Cys-Met-Glu-Arg-Val-Glu-Trp-Leu-Arg-Lys-Leu-Leu-Gln-Asp-Val-NH₂ (SEQ ID NO: 12) which is cyclized between Glu⁶ and Lys¹⁰.
